(19) United States
(12) Patent Application Publication
(10) Pub. No.: US 2009/0136470 A1
(43) Pub. Date: May 28, 2009

(54) REGULATORY T CELLS AND METHODS OF MAKING AND USING SAME

(76) Inventors: HILDE CHEROUTRE, Del Mar, CA (US); YUNJI PARK, San Diego, CA (US); DANIEL DE SOUSA MUCIDA, San Diego, CA (US); IDELFONSO VICENTE-SUAREZ, La Jolla, CA (US)

Correspondence Address: PILLSBURY WINTHROP SHAW PITTMAN LLP, ATTENTION: DOCKETING DEPARTMENT, P.O BOX 10500, McLean, VA, 22102, US

(21) Appl. No.: 12/139,264

(22) Filed: Jun. 13, 2008

Related U.S. Application Data
(60) Provisional application No. 60/943,829, filed on Jun. 13, 2007.
(60) Provisional application No. 60/955,585, filed on Aug. 13, 2007.
(60) Provisional application No. 61/033,282, filed on Mar. 3, 2008.

(51) Int. Cl. A61K 35/12; C12N 5/06; A61K 31/07; C12N 5/08; A61K 31/38

(52) U.S. Cl. 424/93.71; 435/377; 514/725; 435/372.3; 514/432

(57) ABSTRACT

Methods of stimulating or increasing differentiation to regulatory T cells, cultures of regulatory T cells and methods of reducing or decreasing an immune response, inflammation or an inflammatory response, among other things, are provided. Methods include, among other things, contacting blood cells or T cells with an amount of TGF-beta or a TGF-beta analogue and a retinoic acid receptor agonist, or an amount of a retinoid X receptor (RXR) or peroxisome proliferator activated receptor-gamma (PPARgamma) agonist, sufficient to stimulate or increase differentiation to regulatory T cells. Cultures of regulatory T cells include T cells that express a marker associated with regulatory T cells, such as cultures in which regulatory T cells represent, for example, 30% or more of the total number of cells in the culture.

### RELATED APPLICATIONS

This application claims the benefit of priority of U.S. Application Ser. No. 60/943,829, filed Jun. 13, 2007, U.S. Application Ser. No. 60/955,585 filed Aug. 13, 2007, and U.S. Application Ser. No. 61/033,282, filed Mar. 3, 2008, which are expressly incorporated herein by reference.

### GOVERNMENT SPONSORSHIP

This work was supported in part by a grant from National Institutes of Health grant RO1 AI050265-06. The government may have certain rights in the invention.

### TECHNICAL FIELD

The invention relates to regulatory T cells, cultures of regulatory T cells and methods of decreasing, reducing, inhibiting, suppressing, delaying, halting, limiting, controlling, abrogating, eliminating, blocking or preventing an immune response, inflammation or an inflammatory response, methods of decreasing, reducing, inhibiting, suppressing, delaying, halting, limiting, controlling, abrogating, eliminating, blocking or preventing an immune response to an antigen, cell, tissue or organ, among other things. The invention also relates to regulatory T cells, dendritic cells, cultures of regulatory T cells, cultures of dendritic cells, and methods of producing or increasing regulatory T cells, and dendritic cells (e.g., dendritic cells that produce retinoic acid).

### INTRODUCTION

Helper T cells perform critical functions in the immune system through the production of distinct cytokine profiles. In addition to T helper-1 (Th-1) and Th-2 cells, a third subset of polarized effector T cells, characterized by the production of IL-17 and other cytokines—and now called Th-17 cells—is associated with the pathogenesis of several autoimmune conditions. The cytokine, transforming growth factor-beta (TGF-β), converts naïve T cells into regulatory T (Treg) cells which can inhibit autoimmunity and inflammation. TGF-β is a suppressor of Th-1 and Th-2 cell differentiation and drives the conversion of T cells to those with a regulatory phenotype; so called Treg cells. In contrast to the suppression of Th-1 and Th-2 cells, in vitro activation of naïve T cells by dendritic cells (DCs) and TGF-β, together with pro-inflammatory cytokines including IL-6, leads to the differentiation of Th-17 cells. These observations indicate that the priming of T cells by DCs in the presence of TGF-β might lead to opposing immune consequences.

The vitamin A metabolite, retinoic acid (RA) is a key modulator of TGF-β-driven immune deviation capable of suppressing TH-17 differentiation while promoting Foxp3⁺Treg generation. Mucosal dendritic cells (DCs), unique in their capacity to degrade vitamin A to generate RA are able to induce, in the presence of TGF-β, much higher frequency of Foxp3⁺ T cells than splenic DCs. Conversely, in the presence of both IL-6 and TGF-β, while splenic DCs induced high levels of IL-17 producing T cells, mucosal DCs were inefficient inducing these cells. Using RA receptor antagonists and exogenous RA the differential capacity of mucosal DCs to induce Treg versus TH-17 cells was dependent on their RA-production.

Although the two physiological isoforms of retinoic acid (all-trans and 9-cis) are the best characterized in terms of biological function, both retinol and retinal (RAL) have been reported to be able to induce, although inefficiently, gut homing molecules. RAL is also able to inhibit TH-17 differentiation and concomitantly enhance TGF-β mediated Foxp3 induction. Similarly to RA, RAL also directly (APC-free system) inhibited retinoic-acid orphan receptor RORg-t, involved in the TH-17-cell differentiation. Although similar, the functions of RAL seem to have some peculiarities that distinguish it from RA. When added in DC/T cell co-cultures, while RAL suppresses IL-17 production from CD4 cells cultured with either MLN DCs (MDC) or splenic DCs (SDC), the increased induction of Foxp3 by RAL was observed mainly in MDC cultures. These results suggest that in this system, Foxp3 induction could be due to increased RA production when we added the precursor. Consistent with this idea, addition of RAR antagonist LE135 reversed Foxp3 induction, but not IL-17 production.

Although RA can bind to both RAR-RAR homodimers and RAR-RXR heterodimers, RAL does not bind to RAR. Instead, RAL has been shown to bind both RXR and, interestingly, the nuclear receptor PPAR-γ (for peroxisome proliferative activated receptor gamma). This family of nuclear receptors is believed to have many roles in the immune system.

### SUMMARY

The vitamin A metabolite, retinoic acid (RA), is a key regulator of TGF-β-dependent immune responses, capable of inhibiting the IL-6-driven induction of pro-inflammatory Th-17 cells and promoting anti-inflammatory Treg differentiation. Thus, a common metabolite can regulate the balance between pro- and anti-inflammatory immunity.

In accordance with the invention, there are provided methods of stimulating or increasing differentiation to regulatory T cells in vitro, ex vivo and in vivo. In one embodiment, a method includes contacting blood cells or T cells with an amount of TGF-beta or TGF-beta analogue and a retinoic acid receptor agonist, or an amount of a retinoid X receptor (RXR) or peroxisome proliferator activated receptor-gamma (PPARgamma) agonist, sufficient to stimulate or increase differentiation to regulatory T cells. In another embodiment, a method includes contacting blood cells or T cells with an amount of TGF-beta or TGF-beta analogue and a retinoic acid receptor agonist, or an amount of a retinoid X receptor (RXR) or peroxisome proliferator activated receptor-gamma (PPARgamma) agonist, sufficient to increase numbers of regulatory T cells to represent greater than about 30%, 40%, 50%, 60%, 70%, 80%, 90% or 95% of the total number of cells present in a culture, optionally without increasing numbers of regulatory T cells by purification, isolation or proliferation. In particular aspects, T regulatory cells express a marker (e.g., Foxp3, CD103, CCR9, alpha4beta7, CD25 or CTLA4). In additional particular aspects, T cells contacted include naïve T cells or activated T cells.

In accordance with the invention, there are also provided isolated and purified populations and pluralities of regulatory T cells, in which the regulatory T cells express a marker associated with regulatory T cells (e.g., Foxp3, CD103, CCR9, alpha4beta7, CD25 or CTLA4). In one embodiment, regulatory T cells exhibit increased expression of a marker (e.g., CD44) associated with regulatory T cells as compared to expression of the marker in a naïve, activated or effector T cell.

In accordance with the invention, there are further provided cultures (e.g., in vitro and ex vivo) of regulatory T cells that express a marker associated with regulatory T cells (e.g., Foxp3, CD103, CCR9, alpha4beta7, CD25 or CTLA4). In one embodiment, regulatory T cells are in the culture in an amount greater than the amount of regulatory T cells that would be in a culture after contact of blood cells with TGF-beta or a TGF-beta analogue without a retinoid X receptor (RXR) or peroxisome proliferator activated receptor-gamma (PPARgamna) agonist agonist. In another embodiment, regulatory T cells are in the culture in an amount greater than the amount of regulatory T cells that would be in a culture after contact of blood cells with TGF-beta or a TGF-beta analogue without a retinoic acid receptor agonist. In an additional embodiment, in a culture of regulatory T cells, the regulatory T cells represent greater than about 30%, 40%, 50%, 60%, 70%, 80%, 90% or 95% of the total number of cells present in the culture, without numbers of regulatory T cells in the culture being increased by purification, isolation or proliferation. In particular aspects, at least a portion of the regulatory T cells express a marker associated with regulatory T cells (e.g., Foxp3, CD103, CCR9, alpha4beta7, CD25 or CTLA4), have a function associated with regulatory T cells, maintain the differentiated state or survive or proliferate, after introduction into or administration to a subject, for a period of time (e.g., for at least about 8 hours, 12, hours, 16 hours, 24 hours, 48 hours, 72 hours or more, at least about 1, 2, 3, 4, 5, 6, 7, 8, 9, 10, 11, 12, 14, 16, 18, 21, 24, 27, 30 days or more, or at least about 1, 2, 3, 4, 5, 6, 7, 8, 9, 10, 11, 12, 14, 16, 18, 21, 24, 27, 30 or more weeks).

In accordance with the invention, there are additionally provided methods of producing or increasing numbers of regulatory T cells in vitro, ex vivo and in vivo. In one embodiment, a method includes contacting blood cells or T cells with a retinoic acid receptor agonist, or a retinoid X receptor (RXR) or peroxisome proliferator activated receptor-gamma (PPARgamma) agonist, and contacting blood cells or T cells with an antigen (e.g., a self antigen) or an anti-CD3 antibody, in an amount that produces or increases numbers of regulatory T cells. In another embodiment, a method includes contacting blood cells or T cells with a retinoic acid receptor agonist, or a retinoid X receptor (RXR) or peroxisome proliferator activated receptor-gamma (PPARgamma) agonist, in an amount that produces or increases numbers of regulatory T cells.

In accordance with the invention, there are yet further provided methods of inhibiting or decreasing differentiation to activated or effector T cells, and methods of reducing numbers of TH-17+ effector cells. In one embodiment, a method includes contacting T cells with a retinoic acid receptor agonist, or a retinoid X receptor (RXR) or peroxisome proliferator activated receptor-gamma (PPARgamma) agonist, in an amount that inhibits or decreases differentiation to activated or effector T cells. In one embodiment, a method includes contacting TH-17+ effector cells with a retinoic acid receptor agonist, or a retinoid X receptor (RXR) or peroxisome proliferator activated receptor-gamma (PPARgamma) agonist, in an amount that reduces numbers of TH-17+ effector cells.

In accordance with the invention, there are still further provided methods of producing dendritic cells that produce retinoic acid in vitro, ex vivo and in vivo. In one embodiment, a method includes contacting dendritic cells with a retinoic acid receptor agonist, or a retinoid X receptor (RXR) or peroxisome proliferator activated receptor-gamma (PPARgamma) agonist, in an amount that increase production of retinoic acid by the contacted dendritic cells. In particular aspects, dendritic cells include spleen dendritic cells, mucosal dendritic cells, blood, peripheral blood cells, bone marrow monocyte-derived dendritic cells, or inducible dendritic cells (e.g., CD34+ progenitor derived dendritic cells,), CD8− dendritic cells, or CD4−/CD8− dendritic cells.

In accordance with the invention, there are furthermore provided cultures (e.g., in vitro and ex vivo) of dendritic cells. In one embodiment, dendritic cells have been treated with a retinoic acid receptor agonist or a retinoid X receptor (RXR) or peroxisome proliferator activated receptor-gamma (PPARgamma) agonist that stimulates or increases differentiation into regulatory dendritic cells. In another embodiment, dendritic cells have been treated with a retinoic acid receptor agonist or a retinoid X receptor (RXR) or peroxisome proliferator activated receptor-gamma (PPARgamma) agonist that stimulates or increases differentiation into regulatory dendritic cells, and an antigen. In particular aspects, dendritic cells include spleen dendritic cells, mucosal dendritic cells, blood, peripheral blood cells, bone marrow monocyte-derived dendritic cells, or inducible dendritic cells (e.g., CD34+ progenitor derived dendritic cells,), CD8-dendritic cells, or CD4−/CD8− dendritic cells.

In accordance with the invention, there are moreover provided pharmaceutical formulations and kits. In various embodiments, pharmaceutical formulations include regulatory T cells, isolated and purified regulatory T cells, populations and pluralities of regulatory T cells, cultures of regulatory T cells, dendritic cells and dendritic cells that produce retinoic acid, in a pharmaceutically or biologically acceptable carrier or excipient. In various embodiments, kits include regulatory T cells, isolated and purified regulatory T cells, populations and pluralities of regulatory T cells, and cultures of regulatory T cells.

In accordance with the invention, there are yet furthermore provided methods of treating a subject in need of regulatory T cells, or dendritic cells (ex vivo and in vivo). In one embodiment, a method includes administering regulatory T cells, isolated and purified regulatory T cells, populations and pluralities of regulatory T cells, cultures of regulatory T cells, dendritic cells, or dendritic cells that produce retinoic acid, into the subject. In particular aspects, the cells are obtained or derived from cells of the same or a different subject or produced from cells obtained or derived from the same or a different subject. In further particular aspects, the subject has or is at risk of having an undesirable, aberrant or pathologic (acute or chronic) immune response (e.g., an adaptive immune response), inflammatory response, inflammation an autoimmune disease, or has or is at risk of having transplant or graft rejection or graft-versus-host disease.

In accordance with the invention, there are still furthermore provided methods of reducing or decreasing an immune response (e.g., an adaptive immune response), inflammation or an inflammatory response in a subject, either acute or chronic. In one embodiment, a method includes administering a retinoic acid receptor agonist, or a retinoid X receptor (RXR) or peroxisome proliferator activated receptor-gamma (PPARgamma) agonist, to the subject in an amount that reduces or decreases the immune response, inflammation or an inflammatory response in the subject.

In accordance with the invention, there are yet moreover provided methods of reducing or suppressing an immune response to an antigen (a self-antigen or a non-self antigen), cell, tissue or organ in a subject in a subject. In one embodiment, a method includes administering regulatory T cells, a culture of regulatory T cells, dendritic cells, or a culture of dendritic cells, into the subject in an amount that reduces or suppresses the immune response to the antigen (a self-antigen or a non-self antigen), cell, tissue or organ in a subject.

In accordance with the invention, there are still moreover provided methods of reducing or suppressing IL-17 expression or production in a cell, in vitro, ex vivo and in vivo. In one embodiment, a method includes contacting cells with a retinoic acid receptor agonist, or a retinoid X receptor (RXR) or peroxisome proliferator activated receptor-gamma (PPARgamma) agonist, in an amount that reduces or suppresses IL-17 expression or production in the cells.

### DRAWING DESCRIPTIONS

FIGS. 1A-1E: A) shows IL-17 and IFN-γ staining of gated TCR Vβ5⁺CD4⁺ spleen cells from OT-II TCR transgenic mice. CD4⁺CD25⁻ cells were stimulated with OVAp and MLN or spleen (SPL) DCs, and where indicated, with exogenous cytokines and LE135 or all-trans retinoic acid (RA); B) shows IL-17 ELISA of the culture supernatants from 1A, and also with 9-cis retinoic acid (9-cis) (mean±SD); C) shows intracellular IL-17 and IFN-γ staining of gated TCRβ⁺CD8⁺ cells. Total CD8⁺ spleen T cells were stimulated with α-CD3ε and spleen APCs with the indicated cytokines and RA; D) shows RORγt mRNA analyzed at various times by PCR in CD4 T cells stimulated with α-CD3ε and α-CD28. Where indicated, IL-17 inducing cytokines (IL-17 cond.) (black), or these cytokines plus RA (white) were included (mean±SD). Representative data from four studies. E) shows intracellular IL-17 and IFN-γ staining of CD4⁺T cells from small intestine lamina propria 5 days after oral infection with Listeria monocytogenes. Data are representative of 3-4 mice per group.

FIGS. 2A-2D: A) shows intracellular staining of gated TCRβ⁺CD4⁺ cells for IL-17 and IFN-γ of polyclonal CD4⁺CD25⁻ spleen Tcells stimulated with soluble α-CD3, irradiated spleen cells, and with added cytokines and RA as indicated; B) shows intracellular staining of gated TCRβ⁺CD4⁺ cells for IL-17 and IFN-γ of OT-II TCR⁺CD4⁺CD25⁻ spleen T cells stimulated with the relevant OVAp, sorted spleen CD11c⁺DCs and with added cytokines and 9-cis RA (100 nM) as indicated and gated on TCR Vβ5⁺CD4⁺ cells; C) shows intracellular staining of gated TCRβ⁺CD4⁺ cells for IL-17 and IFN-γ of OT-I TCR⁺CD8⁺T cells stimulated with the relevant OVAp and spleen CD11c⁺DCs and without (none) or with the indicated cytokines, without or with RA; D) shows intracellular staining of gated TCRβ⁺CD4⁺ cells for IL-17 and IFN-γ of Polyclonal CD4⁺CD25⁻ spleen T cells stimulated with anti-CD3/CD28 beads and without exogenous cytokines (none) or with the indicated cytokines (IL-17 cond.: TGF-β, IL-6, IL-1β, TNF-α) and with or without RA and gated on TCRβ⁺CD4⁺ cells. Representative data from three studies.

FIGS. 3A-3E: A) shows intracellular staining for Foxp3 and surface CD103 of gated TCR Vβ5⁺CD4⁺ cells from OT-II TCR transgenic mice. CD4⁺CD25⁻T cells were stimulated with OVAp and MLN or SPL DCs, and as indicated, with TGF-β1 and LE135 or RA; B) shows intracellular Foxp3 and CTLA-4 staining of OT-II TCR CD4⁺CD25⁻T cells stimulated as above, except with spleen APCs instead of DC; C) shows CD8⁺T cells from OT-I TCR transgenic mice were stimulated with OVAp and spleen DCs with TGF-β1 and RA. Intracellular staining of gated TCRβ⁺ cells for Foxp3 is shown; D) shows cell surface staining of gated TCRβ⁺CD4⁺ cells for CD103, α₄β₇ and CCR9. CD4⁺CD25⁻T cells were stimulated with soluble α-CD3ε and spleen APCs plus TGF-β1, RA, or TGF-β1 and RA. Isotype controls indicated with solid gray histograms. Representative data from three studies; E) shows a percentage of Foxp3⁺CD4⁺ cells in CD4⁺TCRβ⁺lymphocytes from the small intestine lamina propria 5 days after oral infection with Listeria monocytogenes (left panel) or in naïve controls (right panels). * P<0.05 (test T-student).

FIGS. 4A-4D: A) shows intracellular staining of Foxp3 and CD4 expression by TCRβ⁺ gated T cells isolated from various tissues. sLPL and ILPL indicate small and large intestine lamina propria lymphocytes, respectively, and PLN indicates peripheral lymph node. The numbers represent mean±SEM of the percentage of Foxp3⁺ T cells in the CD4+ T cell population; B) shows intracellular Foxp3 staining and surface staining for CD25 or CD103 of gated TCRβ⁺CD4⁺ T cells. In the lower panels, the numbers indicate the percentage of CD103⁺ cells in the Foxp3⁺ population. Five mice were analyzed for each study; C) shows intracellular staining for Foxp3 and CTLA-4 and surface staining for CD25 of OT-1 TCR⁺CD8⁺T cells stimulated with the relevant OVAp and irradiated spleen APCs for 3 days and without (none) or with the indicated cytokines, and without or with RA. For comparison, OT-II CD4⁺CD25⁻ cells stimulated under the same conditions are also shown; D) shows histograms represent staining of the OT-I CD8⁺ cells, gated on TCRβ⁺CD8⁺ cells, stimulated in the conditions described in (C) for 3, 4 and 5 days. Solid grey—none; grey line-RA; dashed line—TGF-β; black line—TGF-b+RA. Representative data from two studies.

FIGS. 5A-5D: A) shows intracellular staining for Foxp3 and CD103 of OT-II TCR⁺CD4⁺CD25⁻ spleen T cells stimulated with the relevant OVAp, sorted spleen CD11c⁺ DCs and without exogenous cytokines (none) or with indicated cytokines, and without or with RA or 9-cis RA (both at 100 nM). Gated on TCR Vβ5⁺CD4⁺ cells. Representative data from four studies; B) shows intracellular staining of Foxp3 and CD4 staining of naïve polyclonal CD4⁺CD25⁻ spleen T cells stimulated with soluble α-CD3ε, irradiated spleen cells and without exogenous cytokines (none) or with TGF-β2 or TGF-β3 and without or with RA. Representative data from two studies; C) shows the percentage of Foxp3⁺CD4⁺ lymphocytes in total CD4⁺ TCRβ⁺T cells isolated from the spleen of mice 5 days after oral infection with Listeria monocytogenes. Each group received 2 i.p. injections (days 0 and 2) with vehicle, RA or LE540. Data of naïve mice that received 2-week of gavage treatment with vehicle, RA or LE540 are shown on the right side (mean±SD); D) shows intracellular staining for Foxp3 of OT-II CD4 T cells stimulated in the same conditions as described in 5A. Representative data of two studies.

FIGS. 6A-6E: A) shows hematoxylin and eosin staining of distal colon of RAG-1^{-/-} mice 6-7 weeks after co-transfer of 5×10⁵ CD4⁺CD45RB^{hi} cells with 2.5×10⁵ CD4⁺T cells stimulated in vitro with α-CD3ε alone (none) or with TGF-β1 and RA. Original magnification, 40×. Representative data from 4 mice in each group; B) shows body weight of RAG-1^{-/-} mice after transfer of 5×10⁵ CD4⁺CD45RB^{hi} cells with 2.5×10⁵ α-CD3ε stimulated CD4⁺T cells with no additions (squares), TGF-β1 (triangles), or TGF-β1 and RA (diamonds). The mean±SD weight of four animals per group is shown. Data are representative of three studies; C) shows histological scores of the groups in 6B; D) shows Foxp3 intracellular staining of naïve TCRβ⁺CD4⁺ that were initially stimulated with soluble α-CD3ε and spleen APCs with the indicated cytokines. The cells were rested for two days with IL-2, and re-stimulated in the absence of exogenous cytokines before analysis; E) shows intracellular staining for IL-17 of naïve CD4⁺T cells initially stimulated and rested as described in 6D, but in the presence of TGF-β and IL-6, and re-stimulated in the indicated conditions. Percentage of IL-17⁺ cells in the gated TCRβ⁺CD4⁺ cells is depicted. Representative data from three studies.

FIG. 7: shows intracellular staining for IL-17 and IFN-γ of IELs from large intestine isolated from RAG−/− recipient mice, 6-7 weeks after transfer of 5×105 Ly5.2+ (Ly 5.1⁻) CD4⁺CD45RB^{hi} cells together with 2.5×105 CD4⁺T cells (Ly5.1⁺) stimulated in vitro with α-CD3ε alone or together with TGF-β1 or TGF-β1 and RA. Gated on CD4⁺ lymphocytes. Representative data from two studies with 3-4 mice per group.

FIGS. 8A-8E: A) shows CFSE labeled naïve CD4⁺T cells were stimulated with α-CD3ε, spleen APCs, the indicated cytokines and as indicated, with RA. TNF-α, IL-1-β, TGF-β and IL-6, were used to drive IL-17 differentiation. Intracellular staining of gated TCRβ⁺CD4⁺ cells for Foxp3 and IL-17 is depicted; B) shows intracellular staining for Foxp3 and IL-17 of CD8⁺T cells stimulated with soluble α-CD3ε and spleen APCs under the indicated conditions; C) and D) show intracellular staining for Foxp3 and surface staining for CD103 (C) or for intracellular IL-17 and IFN-γ (D) of naïve CD4⁺T cells from B7.1/2^{-/-} IL-2^{+/+} or B7.1/2^{-/-} IL-2^{-/-} mice. Cells were stimulated with soluble α-CD3ε, spleen APCs and the indicated cytokines, RA and/or blocking antibody to IL-2 (20 μg/ml), gated on TCRβ³⁰ CD4⁺ cells; E) shows ELISA for IL-17 in the supernatant of the cultures in 8C and 8D (mean±SD). Representative data of two studies.

FIGS. 9A-9C: A) shows intracellular staining for Foxp3 and IL-17 of polyclonal CD4⁺CD25⁻ T cells stimulated with soluble α-CD3ε, irradiated spleen APCs and TGF-β (5 ng/ml) without or together with indicated concentrations of IL-6 and RA, gated on TCRβ⁺CD4⁺ cells; B) shows intracellular staining for IL-17 and IFN-γ of total CD8⁺T cells from C57BL/6 mice stimulated with soluble α-CD3ε, irradiated spleen APCs and without (none) or with the indicated cytokines and/or RA (100 nM) and/or blocking anti-IL-2 antibodies (20 μg/ml); C) shows ELISA for IL-17 in the supernatants of the cultures set up as described in 9B with the conditions indicated. Representative data of two studies.

FIGS. 10A-10B: show expression of mRNA, as measured by qPCR, for the RALDH enzyme isoforms 1, 2 and 3 (A) or only RALDH2 (B) by sorted total splenic CD11c+ DCs(A) or CD11c+DCs sorted in subpopulations that express CD4, CD8 or plasmacytoid DCs (B).

FIG. 11: shows that retinal enhances Treg differentiation.

FIG. 12: shows that retinal is a suppressor of TH-17 differentiation.

FIG. 13: shows that inhibition of RALDH activity by citral does not reverse RAL effects on TH-17 differentiation.

FIG. 14: shows IL-17 levels under the indicated conditions.

FIG. 15: shows relative expression of Foxp and RORγ under the indicated conditions.

### DETAILED DESCRIPTION

The invention provides, among other things, methods of activating, stimulating, increasing, inducing, enhancing or promoting differentiation to regulatory T cells, in vitro, ex vivo or in vivo. In one embodiment, a method includes contacting blood cells or T cells with an amount of TGF-beta (or a TGF-beta isoform, derivative or analogue) and a retinoic acid receptor agonist, or an amount of a retinoid X receptor (RXR) or peroxisome proliferator activated receptor-gamma (PPARgamma) agonist, sufficient to activate, stimulate, increase, induce, enhance or promote differentiation to regulatory T cells.

The invention also provides, among other things, methods of producing or increasing numbers of regulatory T cells. In one embodiment, a method includes contacting blood cells or T cells with a retinoic acid receptor agonist, or a retinoid X receptor (RXR) or peroxisome proliferator activated receptor-gamma (PPARgamma) agonist, in an amount to produce or increase numbers of regulatory T cells. In particular aspects, the cells are contacted with an antigen (e.g., a self antigen) or an anti-CD3 antibody. In additional particular aspects, the cells are further contacted with IL-2 or not contacted with IL-2.

Regulatory T cells, produced in the absence or presence of an antigen, can be used to provide a subject with tolerance to an antigen. Thus, a subject that has developed or is at risk of developing an undesirable or aberrant immune response against an antigen, such as a self antigen, can be administered regulatory T cells in order to provide antigen tolerance to the subject.

Regulatory T cells (abbreviated as “Tregs,” and also known as suppressor T cells) suppress activation of certain pro-inflammatory components of the immune system in order to maintain immune system homeostasis and tolerance to self-antigens. Genetic deficiencies in regulatory T cells lead to various autoimmune disorders.

Regulatory T cells can be characterized by greater or less expression of certain markers. For example, regulatory T cells express certain markers (e.g., Foxp3 (forkhead box p3), CD4, CD25, CD44, CD103, CCR9, alpha4beta7, IL-2 receptor, CTLA-4 (cytotoxic T-lymphocyte associated molecule-4), CD8, etc.), while expressing less of certain markers (e.g., CD45), as compared to other T cell types (e.g., naïve, activated or effector T cells).

T cells further include, for example, naïve, activated effector (cytotoxic, helper) or memory T cells, and NK T cells. Naïve, activated and effector (cytotoxic, helper) or memory T cells, and NK T cells can also be characterized by greater or less expression of certain cell markers. For example, activated effector T cells do not detectably express Foxp3.

T cells also include a mixed population or plurality of cells, or cells which have enriched therein certain subtypes of cells, including T cells. Thus, T cells can include one or more different T cell types (e.g., regulatory, naïve, activated effector (cytotoxic, helper), memory T cells, NK cells, etc.), B cells, monocytes, macrophages, dendritic cells, red blood cells, etc.

Naïve T cells or activated effector T cells can be converted to regulatory T cells by contact with a retinoic acid receptor agonist, or contact with a retinoid X receptor (RXR) or peroxisome proliferator activated receptor-gamma (PPARgamma) agonist, in vitro, ex vivo or in vivo. The invention methods therefore include decreasing, reducing, inhibiting, suppressing, limiting, controlling, abrogating, eliminating, blocking or preventing activated or effector T cells (e.g., TH-17+ cells), as well as decreasing or, reducing numbers of activated or effector T cells (e.g., TH-17+ cells), in vitro, ex vivo and in vivo.

In accordance with the invention, there are further provided, among other things, methods of inhibiting or decreasing differentiation to activated or effector T cells, and methods of reducing numbers of effector T cells (e.g., TH-17+ cells). In one embodiment, a method includes contacting T cells with a retinoic acid receptor agonist, or a retinoid X receptor (RXR) or peroxisome proliferator activated receptor-gamma (PPARgamma) agonist, in an amount that inhibits or decreases differentiation to activated or effector T cells. In another embodiment, a method includes contacting effector T cells (e.g., TH-17+ cells) with a retinoic acid receptor agonist, or a retinoid X receptor (RXR) or peroxisome proliferator activated receptor-gamma (PPARgamma) agonist, in an amount that reduces numbers of effector T cells (e.g., TH-17+ cells). In additional particular aspects, the cells are further contacted with IL-2 or not contacted with IL-2.

TGF-beta and TGF-beta isoforms, derivatives and analogues are useful in accordance with the invention. Non-limiting examples of TGF-beta isoforms include, for example, TGF-β2, TGF-β1,2, TGF-β3, TGF-β2,3, TGF-β4, and TGF-β5. Non-limiting examples of TGF-beta derivatives include, for example, Non-limiting examples of TGF-beta analogues include, for example, additional TGF-beta isoforms, derivatives and analogues would be known to the skilled artisan.

TGF beta receptors include TGF-beta receptor I (53 kDa) and TGF-beta receptor II (70/80 kDa). TGF-beta can therefore be substituted with molecules that bind to TGF-beta receptor and have a similar agonist activity as TGF-beta, which are referred to as TGF-beta receptor agonists.

The term “modulate” means any change in an activity, function or expression, for example, to stimulate, increase, induce, enhance or promote activity or expression, or to decrease, reduce, inhibit, suppress, delay, halt, limit, control, abrogate, eliminate, block, or prevent an activity, function or expression.

An agonist refers to stimulating, increasing, inducing, enhancing or promoting an activity or expression in vitro, ex vivo or in vivo. An antagonist refers to decreasing, reducing, inhibiting, suppressing, delaying, halting, limiting, controlling, abrogating, eliminating, blocking, or preventing an activity, function or expression in vitro, ex vivo or in vivo.

Retinoic acid receptor agonists include any molecule that activates, stimulates induces, enhances or promotes a retinoic acid receptor activity or function in vitro, ex vivo or in vivo. Non-limiting examples of retinoic acid receptor agonists applicable in the compositions and methods include vitamin A, and vitamin A derivatives, analogues and metabolites. Non-limiting examples of vitamin A metabolites include retinoic acid, and retinoic acid derivatives, analogues and isomers. Non-limiting examples of retinoic acid receptor derivatives include an esters and amides, such as fenretinide and retinaldehyde. Non-limiting examples of retinoic acid receptor analogues include 9-cis retinoic acid, 13-cis retinoic acid and all trans retinoic acid. Non-limiting examples of retinoic acid receptor isomers include an arotinoid, such as adapalene and tazarotene.

Retinoid X receptor (RXR) and peroxisome proliferator activated receptor-gamma (PPARgamma) agonists include any molecule that activates, stimulates induces, enhances, increases or promotes an activity or function of retinoid X receptor (RXR) or peroxisome proliferator activated receptor-gamma (PPARgamma) in vitro, ex vivo or in vivo. Non-limiting examples of a retinoid X receptor (RXR) or peroxisome proliferator activated receptor-gamma (PPARgamma) agonist applicable in the compositions and methods are retinal, and retinal derivatives, stereoisomers, analogues and metabolites. Non-limiting examples of retinal derivatives stereoisomers, analogues and metabolites are all-trans, 13-cis, 11-cis, 9-cis, 7-cis, 11,13-cis, 9,13-cis vitamin A aldehyde, and hydrate, hemiacetal and acetal forms. Non-limiting examples of retinal derivatives include stereoisomers, analogues and metabolites, such as retinal hydrate; retinal methyl hemiacetal; retinal ethyl hemiacetal; retinal propyl hemiacetal; retinal isopropyl hemiacetal; retinal butyl hemiacetal; retinal pentyl hemiacetal; retinal octyl hemiacetal; retinal benzyl hemiacetal; retinal dimethyl acetal; retinal diethyl acetal; retinal dipropyl acetal; retinal diisopropyl acetal; retinal dibutyl acetal; retinal dipentyl acetal; retinal dioctyl acetal; retinal dibenzyl acetal; retinal propylene glycol hemiacetal or acetal; retinal 1,2-O-isopropylidene glyceryl hemiacetal or acetal; retinal 3-allyloxy-1,2-propanediol hemiacetal or acetal; retinal phythyl hemiacetal; retinal diphytyl acetal; retinal dodecyl hemiacetal; and retinal didodecyl acetal. Additional non-limiting examples of retinal derivatives are 5,6-dioxo-5,6-seco-retinal, 5,6-dihydro-5,6-epoxy-retinal, and 4-oxoretinal.

The term “contact” or “contacting” means direct physical contact or interaction or indirect contact or interaction between one entity (e.g., blood cells or T cells or dendritic cells), and another (e.g., TGF-beta, retinoic acid receptor agonist, retinoid X receptor (RXR) agonist or peroxisome proliferator activated receptor-gamma (PPARgamma) agonist). An example of indirect contact or interaction is binding to an intermediary, which in truns binds to a referenced entity. Thus, for example, TGF-beta or retinoic acid contacts or interacts with (e.g., binds) to an entity that in turn contacts or interacts with a cell (e.g., via a TGF-beta or retinoic acid receptor agonist). Thus, a molecule that contacts blood cells or T cells may or may not physically contact or interact with the cells, but may bind to an intermediary molecule that, in turn, contacts or interacts with the cells.

Blood cells include peripheral blood mononuclear cells (PBMCs), whole blood, or subsets or populations of cells that include one or more cells types in the blood cells. Subsets include, for example, lymphocytes (e.g., T cells, natural killer or NK cells) and monocytes (e.g., macrophages, dendritic cells).

Blood cells may be animal cells, such as mammalian cells. Mammalian cells include primate cells (e.g., human, ape, gibbon, gorilla, chimpanzee, orangutan, macaque, etc.), domestic animal cells (dogs and cats), farm animal cells (chickens, ducks, horses, cows, goats, sheep, pigs), and experimental animal cells (mouse, rat, rabbit, guinea pig).

Blood cells and T cells may be obtained directly from a subject, or derived from cells obtained from a subject, e.g., progeny cells from one or more cell doublings of parental cells obtained from a subject. Thus, for example, blood or T cells may be from stored or frozen cells, or derived from a culture of cells. Blood cells or T cells from a subject may be treated in accordance with an invention method and can be further manipulated, proliferated or stored (e.g., frozen), if desired. Treated cells and cell cultures can optionally be re-introduced back into the same subject (autologous transplant) or a different subject (such as a subject from the same species, i.e., allogeneic transplantation, or a different species xenotransplant), for example, in order to effect a treatment method as set forth herein.

The invention also provides, among other things, in vitro and ex vivo cultures of T cells. In one embodiment, a culture of regulatory T cells that express a marker associated with regulatory T cells (e.g., oen or more of Foxp3, CD103, CCR9, alpha4beta7, CD25, CTLA4, etc.) is provided. In another embodiment, in a culture of regulatory T cells the regulatory T cells represent greater than about 30%, 40%, 50%, 60%, 70%, 80%, 90% or 95% of the total number of cells present in the culture. In a particular aspect, the regulatory T cells are in the culture in an amount greater than the amount of regulatory T cells in a culture after contact of blood cells with TGF-beta without (i.e., in the absence of exogenous) a retinoid X receptor (RXR) or peroxisome proliferator activated receptor-gamma (PPARgamma) agonist agonist. In another particular aspect, the regulatory T cells are in the culture in an amount greater than the amount of regulatory T cells in a culture after contact of blood cells with TGF-beta without a retinoic acid receptor agonist. In a further particular aspect, in a culture of regulatory T cells the regulatory T cells represent greater than about 30%, 40%, 50%, 60%, 70%, 80%, 90% or 95% of the total number of cells present in the culture without increasing the numbers of regulatory T cells in the culture by purification, isolation or proliferation. In an additional particular aspect, in a culture of regulatory T cells the regulatory T cells represent greater than about 30%, 40%, 50%, 60%, 70%, 80%, 90% or 95% of the total number of cells present in the culture by increasing the numbers of regulatory T cells by differentiation of naïve or activated T cells (e.g., conversion of effector T helper cells to regulatory T cells). Such cells can be produced in accordance with the invention methods.

In vitro and ex vivo cultures of T cells include T cells in which at least a portion of the regulatory T cells maintain the differentiated state, or survive or proliferate for a period of time, or after introduction into or administration to a subject in vivo. In vitro and ex vivo cultures of T cells also include T cells in which at least a portion of the regulatory T cells express a marker associated with regulatory T cells (e.g., Foxp3, CD103, CCR9, alpha4beta7, CD25, CTLA4, etc.), survive or proliferate for a period of time, or after introduction into or administration to a subject in vivo. In vitro and ex vivo cultures of T cells further include T cells in which at least a portion of the regulatory T cells have a function associated with regulatory T cells (e.g., decrease, reduce, inhibit, suppress, delay, halt, limit, control, abrogate, eliminate, block, or prevent an immune response, inflammation or an inflammatory response, tropism to a particular tissue or organ, etc.) for a period of time, or after introduction into or administration to a subject in vivo. In particular aspects, an in vitro or ex vivo culture of T cells include at least a portion of T cells that maintain the differentiated state, express a marker associated with regulatory T cells, or have a function associated with regulatory T cells, for at least about 8 hours, 12, hours, 16 hours, 24 hours, 48 hours, 72 hours or more, about 1, 2, 3, 4, 5, 6, 7, 8, 9, 10, 11, 12, 14, 16, 18, 21, 24, 27, 30 days or more, or about 1, 2, 3, 4, 5, 6, 7, 8, 9, 10, 11, 12, 14, 16, 18, 21, 24, 27, 30 or more weeks, or after introduction into or administration to a subject for at least about 8 hours, 12, hours, 16 hours, 24 hours, 48 hours, 72 hours or more, or about 1, 2, 3, 4, 5, 6, 7, 8, 9, 10, 11, 12, 14, 16, 18, 21, 24, 27, days or more, or about 1, 2, 3, 4, 5, 6, 7, 8, 9, 10, 11, 12, 14, 16, 18, 21, 24, 27, 30 or more weeks in vivo.

Such cultures of regulatory T cells can decrease, reduce, inhibit, suppress, delay, halt, limit, control, abrogate, eliminate, block, or prevent an undesired or aberrant immune response, inflammation or an inflammatory response. Thus, in various embodiments, regulatory T cells provide, among other things, increased, or greater inhibition, reduction or suppression of an immune response, inflammation or an inflammatory response in a subject for a period of time (e.g., 8 hours, 12, hours, 16 hours, 24 hours, 48 hours, 72 hours or more, or about 1, 2, 3, 4, 5, 6, 7, 8, 9, 10, 11, 12, 14, 16, 18, 21, 24, 27, 30 days or more, or about 1, 2, 3, 4, 5, 6, 7, 8, 9, 10, 11, 12, 14, 16, 18, 21, 24, 27, 30 or more weeks) than in the absence of such regulatory T cells. In additional various embodiments, regulatory T cells can also provide, among other things, greater regulatory T cell function after introduction into or administration to a subject for a period of time (e.g., 8 hours, 12, hours, 16 hours, 24 hours, 48 hours, 72 hours or more, or about 1, 2, 3, 4, 5, 6, 7, 8, 9, 10, 11, 12, 14, 16, 18, 21, 24, 27, 30 days or more, or about 1, 2, 3, 4, 5, 6, 7, 8, 9, 10, 11, 12, 14, 16, 18, 21, 24, 27, 30 or more weeks) than regulatory T cells produced by contact of blood cells with TGF-beta in the absence of an retinoid X receptor (RXR) or peroxisome proliferator activated receptor-gamma (PPARgamma) agonist, or greater regulatory T cell function after introduction into or administration to a subject for a period of time (e.g., 8 hours, 12, hours, 16 hours, 24 hours, 48 hours, 72 hours or more, or about 1, 2, 3, 4, 5, 6, 7, 8, 9, 10, 11, 12, 14, 16, 18, 21, 24, 27, 30 days or more, or about 1, 2, 3, 4, 5, 6, 7, 8, 9, 10, 11, 12, 14, 16, 18, 21, 24, 27, 30 or more weeks) than regulatory T cells produced by contact of blood cells with TGF-beta in the absence of a retinoic acid receptor agonist.

The invention further provides, among other things, cultures of dendritic cells. In one embodiment, a culture of dendritic cells (e.g., CD8− or CD4−/CD8−) is treated with a retinoic acid receptor agonist or a retinoid X receptor (RXR) or peroxisome proliferator activated receptor-gamma (PPARgamma) agonist in an amount that stimulates or increases differentiation into regulatory dendritic cells. In another embodiment, a culture of dendritic cells (e.g., CD8− or CD4−/CD8−) is treated with a retinoic acid receptor agonist or a retinoid X receptor (RXR) or peroxisome proliferator activated receptor-gamma (PPARgamma) agonist and an antigen. In a particular aspect, a culture of dendritic cells is further contacted or treated with an antigen.

The invention moreover provides, among other things, methods of producing dendritic cells that produce retinoic acid. In one embodiment, a method includes contacting dendritic cells with a retinoic acid receptor agonist, or a retinoid X receptor (RXR) or peroxisome proliferator activated receptor-gamma (PPARgamma) agonist, in an amount that increases production of retinoic acid by the contacted dendritic cells.

Dendritic cells include spleen dendritic cells, mucosal dendritic cells, blood, peripheral blood cells, bone marrow dendritic cells, monocyte-derived dendritic cells, or inducible dendritic cells including, for example, CD34+ progenitor derived dendritic cells. Dendritic cells include CD8− dendritic cells, and CD4−/CD8− dendritic cells. Contacted dendritic cells can exhibit increased or stimulated expression of retinaldehyde dehydrogenase (RALDH2), as compared to dendritic cells not contacted with exogenous retinoic acid receptor agonist, or a retinoid X receptor (RXR) or peroxisome proliferator activated receptor-gamma (PPARgamma) agonist.

Dendritic cells can be obtained from a subject, or derived from cells that were at obtained from a subject, e.g., progeny cells of one or more cell doublings of parental cells obtained from a subject. Thus, for example, dendritic cells may be from stored or frozen cells, or derived from a culture of cells. Dendritic cells can be induced from inducing pluripotent stem (iPS) cells (e.g., Yamanaka et al., WO 2007/069666) Dendritic cells treated according to the invention can be further manipulated, proliferated or stored (e.g., frozen), if desired. Treated cells and cell cultures can optionally be re-introduced back into the same subject (autologous transplant) or a different subject (such as a subject from the same species, i.e., allogeneic transplantation, or a different species xenotransplant), for example, in order to effect a treatment method as set forth herein.

Contacting and treatment as used herein includes in solution, in solid phase, in culture, in vitro, ex vivo, in a cell, organ or tissue, and in vivo. Contacting and treatment in vivo can be referred to as administering, administration or delivery. Accordingly, methods embodiments include methods of contact, treatment, administration and delivery, in vitro (in solution in solid phase or in culture), ex vivo and in vivo.

Methods can modulate, among other things, T cell proliferation, differentiation or development, or a T cell function or activity, for example. T cell functions and activities that can be modulated in accordance with the invention include, for example, T cell cytotoxicity, T cell tropism to a particular tissue or organ, T cell cytokine, chemokine or marker expression or secretion, or T cell cytokine or chemokine receptor expression or secretion.

Methods embodiments, including, for example, treatment methods, are applicable to treating any physiological condition, disorder, illness, disease and symptom or pathology thereof potentially amenable to treatment by administering or contact with regulatory T cells, TGF-beta (or a TGF-beta isoform, derivative or analogue), retinoic acid receptor agonist, a retinoid X receptor (RXR) or peroxisome proliferator activated receptor-gamma (PPARgamma) agonist, or dendritic cells ex vivo or in vivo. The methods embodiments therefore include treatment of subjects generally in need of or that could benefit from regulatory T cells, TGF-beta, retinoic acid receptor agonist, retinoid X receptor (RXR) or peroxisome proliferator activated receptor-gamma (PPARgamma) agonist, as well as subjects having particular physiological conditions, disorders, illnesses, diseases and symptoms and pathologies thereof, such as undesirable and aberrant immune responses (e.g., acute or chronic inflammation, inflammatory responses, and autoimmune disorders).

The invention therefore additionally provides, among other things, compositions and methods for treating a physiological condition, disorder, illness, disease, or a symptom or pathology thereof that may respond to regulatory T cells or cultures of regulatory T cells, may respond to increasing, stimulating, inducing, enhancing or promoting T regulatory cell differentiation or production, cultures of dendritic cells, dendritic cell differentiation or production, or may respond to TGF-beta (or a TGF-beta isoform, derivative or analogue), retinoic acid receptor agonist, a retinoid X receptor (RXR) or peroxisome proliferator activated receptor-gamma (PPARgamma) agonist, ex vivo or in vivo. In one embodiment, a method includes administering an amount of TGF-beta (or a TGF-beta isoform, derivative or analogue) and a retinoic acid receptor agonist, or an amount of a retinoid X receptor (RXR) or peroxisome proliferator activated receptor-gamma (PPARgamma) agonist, sufficient to treat the subject. In another embodiment, a method includes administering regulatory T cells in an amount sufficient to treat the subject. In a further embodiment, a method includes administering dendritic cells in an amount sufficient to treat the subject.

Methods embodiments include treating physiological conditions, disorders, illnesses, diseases, and symptoms or pathologies caused by or associated with physiological conditions, disorders, illnesses, and diseases. In particular embodiments, a method includes administering or contact ex vivo or in vivo with, for example, regulatory T cells, dendritic cells, TGF-beta (or a TGF-beta isoform, analogue or derivative), retinoic acid receptor agonist, or a retinoid X receptor (RXR) or peroxisome proliferator receptor gamma (PPAR-gamma) agonist. Treating subjects by administering or contact with, for example, regulatory T cells, dendritic cells, TGF-beta (or a TGF-beta isoform, analogue or derivative), retinoic acid receptor agonist, or a retinoid X receptor (RXR) or peroxisome proliferator receptor gamma (PPAR-gamma) agonist, are therefore also included.

The term “treatment” refers to contact or administration to a subject. The term “therapeutic,” when used in reference to treatment, means that the treatment is practiced on a subject that has or is at risk of having a physiological condition, disorder, illness or disease, or exhibits one or more symptoms or pathologies associated with or caused by the physiological condition, disorder, illness or disease, in which a beneficial effect is desired to be provided. Therapeutic treatment methods are therefore intended to provide an objective or subjective (perceived) effect or benefit, e.g., an improvement in one or more symptoms or pathologies associated with or caused by a physiological condition, disorder, illness, or disease.

“Prophylaxis” and grammatical variations thereof refer to contact, administration or in vivo delivery to a subject prior to a known or established physiological condition, disorder, illness, disease, or a symptom or pathology thereof. Prophylactic situations include those where it is not known if a subject has the physiological condition, disorder, illness, disease, or a symptom or pathology thereof. In such a method, the effect of contact with, administration, ex vivo or in vivo delivery of regulatory T cells, dendritic cells, retinoic acid receptor agonist, TGF-beta (or a TGF-beta isoform, derivative or analogue), or a retinoid X receptor (RXR) or peroxisome proliferator receptor gamma (PPAR-gamma) agonist, can be to decrease, reduce, inhibit, suppress, halt, limit, control, abrogate, eliminate, block, or prevent probability of or susceptibility towards developing a physiological condition, disorder, illness, disease, or a symptom or pathology caused by or associated with a physiological condition, disorder, illness, or disease.

As is typical for any treatment or therapy, different subjects will exhibit different responses to treatment and some may not respond or respond less than desired to a particular treatment method. For example, due to variability in responsiveness, not all subjects will respond to a given treatment or therapeutic method.

Non-limiting physiological conditions, disorders, illnesses, diseases, and symptoms or pathologies can be caused by or associated with insufficient, deficient, decreased, or reduced numbers, activity or differentiation of regulatory T cells or dendritic cells (antigen specific or not antigen specific). The methods embodiments therefore include treatment of subjects generally in need of regulatory T cells or dendritic cells, and any physiological condition, disorder, illness, disease, symptom or pathology thereof that is caused by or results in insufficient numbers, activity or differentiation, deficient, decreased, or reduced numbers, activity or differentiation, or loss of regulatory T cells or dendritic cells.

Additional non-limiting examples include physiological conditions, disorders, illnesses, diseases and symptoms and pathologies thereof caused by undesirable numbers or activity of activated or effector T cells or dendritic cells (antigen specific or not antigen specific). The methods embodiments therefore include treatment of subjects generally in need of or that may benefit from decreased, reduced, inhibited, suppressed, delayed, halted, limited, control, abrogated, eliminated, blocked, or prevent activated or effector T cells or dendritic cells, and any physiological condition, disorder, illness, disease, symptom or pathology thereof that is caused by or results in undesirable numbers or activity, or increased, enhanced, stimulated, promoted or induced numbers or activity of activated or effector T cells or dendritic cells.

The invention additionally provides, among other things, methods of reducing or decreasing an immune response, inflammation or an inflammatory response in a subject. In one embodiment, a method includes contacting or administering a retinoic acid receptor agonist, or a retinoid X receptor (RXR) or peroxisome proliferator activated receptor-gamma (PPARgamma) agonist, to a subject in an amount that reduces or decreases the immune response, inflammation or an inflammatory response in the subject.

Method embodiments include treatment of physiological conditions, disorders, illnesses, diseases, or symptoms or pathologies, caused by or associated with undesirable and aberrant immune responses, inflammation, inflammatory responses, immune disorders and immune diseases. In various embodiments, methods include treating chronic and acute forms of undesirable or aberrant inflammatory responses and inflammation immune disorders and immune diseases; treating chronic and acute forms of undesirable or aberrant proliferation, survival, differentiation, death, or activity of a lymphocyte, such as a regulatory, effector or activated T cell. In exemplary methods, a subject is contacted or administered one or more of T regulatory cells, dendrite cells, TGF-beta (TGF beta receptor agonist), a retinoic acid receptor agonist, or a retinoid X receptor (RXR) or peroxisome proliferator activated receptor-gamma (PPARgamma) agonist.

As used herein, an “immune response, inflammation or inflammatory response” refers to any immune response, inflammation or inflammatory response, or activity or function. An undesirable or aberrant immune response, inflammation or inflammatory response is greater or less than desired or physiologically normal. An undesirable immune response, inflammation or inflammatory response can be a normal response, function or activity, that is undesired or inappropriate. Thus, normal immune responses, inflammation and inflammatory responses considered undesirable or inappropriate, even if not aberrant, are included within the meaning of these terms. An undesirable immune response, inflammation or inflammatory response can also be an aberrant response, function or activity. An aberrant immune response, inflammation or inflammatory response is abnormal. Undesirable, inappropriate aberrant or abnormal immune responses, inflammation and inflammatory responses can be humoral, cell-mediated or a combination thereof, either chronic or acute.

A non-limiting example of an undesirable, aberrant or abnormal immune response is where the immune response is hyper-responsive, such as in the case of an autoimmune condition, disorder, illness or disease. Another example of an undesirable, aberrant or abnormal immune response is where an immune response leads to acute or chronic immune response, inflammation or an inflammatory response systemically, regionally or locally, in any tissue or organ. Yet another example of an undesirable, aberrant or abnormal immune response is where an immune response leads to destruction of cells, tissue or organ, such as a cell, tissue or organ transplant, as in transplant rejection or graft vs. host disease (GVHD). Still another example of an undesirable, aberrant or abnormal immune response is where the immune response is directed against a self or non-self antigen, cell, organ or tissue where response to an antigen is greater than desired or is aberrant.

The terms “immune disorder” and “immune disease” mean an immune function or activity that is greater than (e.g., autoimmunity) or less than (e.g., immunodeficiency) desired or is abnormal. Immune disorders and diseases can be characterized by different physiological symptoms or abnormalities, depending upon the disorder or disease.

Particular non-limiting examples of immune disorders and diseases to which the methods embodiments apply include autoimmune disorders and immunodeficiencies. Methods embodiments for treating autoimmune conditions, disorders, illnesses, diseases or symptoms are therefore provided.

Autoimmune disorders are generally characterized as an undesirable, aberrant or abnormal increased or inappropriate response, activity or function of the immune system. Exemplary non-limiting autoimmune disorders treatable according to the invention include multiple sclerosis (MS), diabetes mellitus types I or II, rheumatoid arthritis (RA), juvenile rheumatoid arthritis, osteoarthritis, psoriatic arthritis, encephalomyelitis, myasthenia gravis, systemic lupus erythematosus (SLE), autoimmune thyroiditis, atopic dermatitis, eczematous dermatitis, psoriasis, Sjögren's Syndrome, intestinal inflammation, inflammatory diseases of the gastrointestinal tract (e.g., Crohn's disease, ulcerative colitis, inflammatory bowel disease (IBD), Celiac disease, and other gastrointestinal inflammatory diseases), aphthous ulcer, iritis, conjunctivitis, keratoconjunctivitis, asthma, allergic asthma, cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, polymyositis, Wegener's granulomatosis, hepatitis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, immune-mediated infertility, autoimmune Addison's disease, pemphigus vulgaris, pemphigus foliaceus, dermatitis herpetiformis, autoimmune alopecia, Vitiligo, autoimmune hemolytic anemia, pernicious anemia, Guillain-Barre syndrome, Stiff-man syndrome, acute rheumatic fever, sympathetic ophthalmia, Goodpasture's syndrome, systemic necrotizing vasculitis, primary biliary cirrhosis and myelodysplastic syndrome.

Additional examples of immune conditions, disorders, illnesses, diseases and symptoms to which the methods apply include chronic and acute inflammation and inflammatory responses. Inflammation and inflammatory responses are generally characterized as undesirable, aberrant or abnormal increased or inappropriate inflammatory response, or an activity or function of the immune system that causes or is associated with inflammation.

Exemplary inflammatory responses and inflammation treatable in accordance with the invention include inflammatory responses and inflammation caused by or associated with proliferation, survival, differentiation, death or activity of T cells (e.g., activated effector T cells, or regulatory T cells) antigen presenting cells (e.g., dendritic cells) or B cells. Methods (e.g., treatment) include decreasing, reducing, inhibiting, suppressing, delaying, limiting, controlling, abrogating, eliminating, blocking, or preventing occurrence, progression, severity, frequency or duration of a symptom or characteristic of an immune response, inflammation or an inflammatory response. At the whole body, regional or local level, an immune response, inflammation or an inflammatory response is generally characterized by swelling, pain, headache, fever, nausea, skeletal joint stiffness or lack of mobility, rash, redness or other discoloration, or tissue or cell damage. At the cellular level, an immune response, inflammation or an inflammatory response is characterized by one or more of cell infiltration of the region, production of antibodies (e.g., autoantibodies), production of proinflammatory cytokines, lymphokines, chemokines, interferons or interleukins, cell growth and maturation factors (e.g., differentiation factors), cell proliferation, cell differentiation, cell accumulation or migration and cell, tissue or organ damage. Methods embodiments include treatment at the whole body, regional or local level, as well as at the cellular level.

Undesirable or an aberrant immune response, inflammation or an inflammatory response, mediated by cellular or humoral immunity, may cause, directly or indirectly, cell, tissue or organ damage, either to multiple cells, tissues or organs, or specifically to a single cell type, tissue type or organ. Exemplary tissues and organs that can exhibit damage include epidermal or mucosal tissue, gut, bowel (small or large intestine), pancreas, thymus, liver, kidney, spleen, skin, or a skeletal joint (e.g., knee, ankle, hip, shoulder, wrist, finger, toe, or elbow). Treatment can result in decreasing, reducing, inhibiting, limiting, suppressing, delaying, halting, limiting, controlling, abrogating, eliminating, blocking or preventing progression or worsening of cellular, tissue or organ damage.

Non limiting physiological conditions further include transplants and grafts. A “transplant,” or “graft” and grammatical variations thereof means grafting, implanting, or transplanting a cell, tissue or organ from a part of the body to the same or a different part of the same subject (autologous), or from one individual or animal to another individual or animal (e.g., human or animal allogeneic). The transplanted cell, tissue or organ may therefore be autologous, an allograft or a xenograft. Exemplary transplant cells include neural cells, adult and embryonic stem cells, and bone marrow. Exemplary transplant tissues include skin, blood vessel, muscle, eye. Exemplary transplant organs include heart, lung, liver and kidney. The term also includes genetically modified cells, tissue and organs, e.g., by ex vivo gene therapy in which the transformed cells, tissue and organs are obtained or derived from a subject (e.g., human or animal) and then reintroduced into the same (autologous) or a different subject (e.g., human or animal allogeneic).

Methods of the invention therefore include reducing, decreasing, inhibiting, limiting, suppressing, controlling, preventing or blocking transplant or graft rejection and graft-versus-host disease (GVHD) in a subject. For example, treatment can result in reducing, decreasing, inhibiting, limiting, suppressing, controlling, preventing or blocking damage to a transplanted or grafted cell, tissue or organ, or a cell, tissue or organ of a subject as in GVHD. Such treatment methods can be performed prior to, concurrently with, immediately following or after transplant or grafting of a cell, tissue or organ in a subject.

Methods embodiments also include treatment to increase, stimulate, enhance, promote, and induce, tolerance to an antigen, cell, organ or tissue. Such treatment methods can be performed in order to decrease, reduce, inhibit, suppress, delay, halt, limit, control, abrogate, eliminate, block, or prevent an undesirable or aberrant immune response to an antigen, cell, organ or tissue, such as a self antigen, cell, organ or tissue that leads to or contributes to an acute or chronic inflammatory response, inflammation or an autoimmune condition or disease. Such treatment methods can also be performed in order to decrease, reduce, inhibit, suppress, delay, halt, limit, control, abrogate, eliminate, block, or prevent an undesirable or aberrant immune response to an antigen, such as a non-self antigen, cell, organ or tissue (e.g., an allogeneic graft, cell, tissue or organ transplant).

The invention therefore moreover provides, among other things, methods of reducing or suppressing an immune response to an antigen, cell, tissue or organ in a subject. In one embodiment, a method includes administering regulatory T cells or dendritic cells to a subject in an amount that reduces or suppresses the immune response to the antigen, cell, tissue or organ the subject. In particular aspects, a method treats a subject that has or is at risk of having an undesirable, aberrant or pathologic adaptive immune response, an acute or chronic immune response, an acute or chronic inflammatory response or inflammation, an autoimmune condition or disease, a graft or transplant rejection (e.g., stem cell transplantation, bone marrow transplantation or an organ or tissue transplantation), or graft-versus-host disease.

Methods embodiments further include reducing or suppressing IL-17 expression or production in a cell (e.g., a T cell, such as a CD4+ T cell). In one embodiment, a method includes contacting cells with a retinoic acid receptor agonist, or a retinoid X receptor (RXR) or peroxisome proliferator activated receptor-gamma (PPARgamma) agonist, in an amount that reduces or suppresses IL-17 expression or production in the cells. In particular aspects, the cells (e.g., T cells, such as a CD4+ T cells) are contacted in vitro or in vivo.

As used herein, the term “associated with,” when used in reference to the relationship between a physiological condition, disorder, illness, disease, or symptom, and an effect or consequence of the physiological condition, disorder, illness, disease, symptom, means that the effect or consequence is caused by the physiological condition, disorder, illness, disease, or is a secondary effect or consequence of the physiological condition, disorder, illness, disease, or a symptom or pathology thereof. A symptom that is present in a subject may therefore be a direct result of or caused by the physiological condition, disorder, illness, disease, or a symptom or pathology thereof, or may be an indirect result of the physiological condition, disorder, illness, disease, or a symptom or pathology thereof. For example, certain physiological conditions, disorders, illnesses, diseases, and symptoms and pathologies that occur may be an indirect effect of an undesirable or aberrant immune response, inflammation or an inflammatory response, in the subject.

Methods of the invention include one or more symptoms, pathologies, or side effects of a physiological condition, disorder, illness, disease, symptom or an effect or consequence of the physiological condition, disorder, illness, disease or a symptom or pathology thereof. A symptom, pathology or side effect that is present in a subject may be the direct result of or caused by the physiological condition, disorder, illness or disease, or may be due at least in part to a secondary or subsequent effect, such as the subject reacting or responding to (e.g., an immunological response) the physiological condition, disorder, illness or disease. Such secondary effects are considered to be associated with the condition, disorder, illness, disease or symptom.

Methods embodiments can produce or result in a beneficial effect or improvement in a subjects' physiological condition, disorder, illness, disease or a symptom or pathology thereof. Methods embodiments therefore include, among other things, treatment methods that result in a beneficial effect to a subject. An example of a beneficial effect or improvement is stimulating, increasing, inducing, enhancing or promoting numbers, differentiation into, proliferation or activity of regulatory T cells. Another example of a beneficial effect or improvement is reducing, decreasing, inhibiting, limiting, suppressing, controlling, delaying, abrogating, preventing or blocking numbers, differentiation into, proliferation or activity of activated or effector T cells. An additional example of a beneficial effect or improvement is stimulating, increasing, inducing, enhancing or promoting numbers, differentiation into, proliferation or activity of dendritic cells, such as retinoic acid producing dendritic cells. A further example of a beneficial effect or improvement is reducing, decreasing, inhibiting, limiting, suppressing, controlling, delaying, abrogating, preventing or blocking IL-17 expression or production by cells. Additional beneficial effects include reducing, decreasing, inhibiting, limiting, suppressing, controlling, delaying, abrogating, ameliorating, preventing or blocking onset, progression, severity, duration, frequency or probability of an undesirable or aberrant immune response, inflammation, or an inflammatory response in a subject.

Additional non-limiting examples of a beneficial effect or improvement include decreasing, reducing, inhibiting, suppressing, delaying, halting, limiting, controlling, abrogating, eliminating, blocking, or preventing probability, susceptibility or likelihood that the subject so treated will manifest one or more symptoms or adverse effects of the physiological condition, disorder, illness, or disease. Symptoms caused by or associated with the various physiological conditions, disorders, illnesses, and diseases (e.g., an undesirable or aberrant immune response, inflammation, or inflammatory response, an autoimmune disease or disorder), are set forth herein and would be known to the skilled artisan and, therefore, improvements in any adverse symptom, pathology or physiological or psychological response are included in the various treatment embodiments.

Treatment embodiments also include reducing or eliminating a need, dosage amount or frequency of another treatment, such as another drug or other agent used for treatment. For example, a subject having or at risk of having an undesirable or aberrant immune response, inflammation, or inflammatory response may no longer require or may require less of another treatment for the undesirable or aberrant immune response inflammation, or inflammatory response.

A treatment method or therapeutic method that provides a beneficial effect or improvement need not result in complete ablation of the undesirable or aberrant immune response inflammation, or inflammatory response, or any particular physiological condition, disorder, illness, disease, or symptom or pathology thereof. Rather, a beneficial effect or improvement may be any objective measurable or detectable effect, or any subjective benefit or improvement in the physiological condition, disorder, illness, disease, or symptom or pathology thereof, of a treated subject. A beneficial effect or improvement therefore includes a subjective or objective reduction in the occurrence, frequency, severity, progression, or duration of a physiological condition, disorder, illness, disease, or symptom thereof, including the underlying cause or a consequence of the physiological condition, disorder, illness, disease, or symptom thereof, or a reversal of the physiological condition, disorder, illness, disease, or symptom thereof. A treatment that provides a beneficial effect or improvement, “ameliorate” is used synonymously, therefore need not be a complete ablation of any or all adverse symptoms or complications associated with the physiological condition, disorder, illness, disease, or symptom, but is any measurable or detectable, objectively or subjectively, effect, benefit or improvement in the physiological condition, disorder, illness, disease, or a symptom or pathology thereof. Thus, reducing, inhibiting, decreasing, eliminating, suppressing, delaying, halting, limiting, controlling, preventing or blocking a progression or worsening of the physiological condition, disorder, illness, disease, or a symptom or pathology thereof is a satisfactory outcome.

Stabilizing an undesirable or aberrant immune response, inflammation, or an inflammatory response in a subject is therefore considered a beneficial effect. For example, regulatory T cells may stabilize an undesirable or aberrant immune response, inflammation or an inflammatory response. Dendritic cells may stabilize a hyperproliferative condition or disorder, such as a tumor or cancer. Thus, a treatment is achieved when there is an incremental improvement in the subject's condition or a partial reduction or a stabilization of a physiological condition, disorder, illness, disease, or adverse symptom or pathology thereof, over a short or long duration (hours, days, weeks, months, years, or cure).

In methods embodiments, additional compositions or method steps can be included. In one embodiment, a method further includes proliferating or expanding regulatory T cells or dendritic cells in vitro, ex vivo or in vivo. In another embodiment, a method further includes contacting blood cells or T cells with a TGF-beta agonist in vitro, ex vivo or in vivo. In further embodiments, a method includes contacting blood cells or T cells with interleukin-2 (IL-2) or excluding contacting blood cells or T cells with IL-2 in vitro, ex vivo or in vivo. In another embodiment, a method further includes administering to a subject regulatory T cells, dendritic cells, TGF-beta (or TGF-beta receptor agonist), a retinoic acid receptor agonist, or a retinoid X receptor (RXR) or peroxisome proliferator activated receptor-gamma (PPARgamma) agonist.

In embodiments of the methods in which there is a desired outcome, for example, a treatment or therapeutic method that provides a beneficial effect or improvement to a subject, a composition, e.g., regulatory T cells, dendritic cells, TGF-beta (or a TGF-beta receptor agonist), a retinoic acid receptor agonist, or a retinoid X receptor (RXR) or peroxisome proliferator activated receptor-gamma (PPARgamma) agonist, can be administered in a sufficient or effective amount. As used herein, a “sufficient amount” or “effective amount” or an “amount sufficient” or an “amount effective” refers to an amount that provides, in single or multiple doses, alone or in combination with one or more other compounds, treatments, agents (e.g., a drug) or therapeutic regimens, a long term or a short term detectable, measurable or a desirable subjective or objective outcome for a given subject, of any degree or for any time period or duration (e.g., for minutes, hours, days, months, years, or cured).

A “sufficient amount” or “effective amount” therefore includes decreasing, reducing, inhibiting, suppressing, delaying, halting, limiting, controlling, abrogating, eliminating, blocking or preventing onset; decreasing, reducing, inhibiting, suppressing, delaying, halting, limiting, controlling, abrogating, eliminating, blocking or preventing a progression or worsening of a physiological condition, disorder, illness, disease, or adverse symptom or pathology thereof; or reducing, relieving, ameliorating, or alleviating, severity, frequency, duration, susceptibility or probability of a physiological condition, disorder, illness, disease, or symptom. In addition, hastening a subject's recovery from a physiological condition, disorder, illness, disease, or symptom or pathology thereof is considered a sufficient or effective amount. Various beneficial effects and indicia of therapeutic and prophylactic benefit are as set forth herein and would be known to the skilled artisan.

Amounts, frequencies or duration also considered sufficient and effective are those that result in the elimination or a reduction in amount, frequency or duration of another compound, agent, treatment or therapeutic regimen. For example, a treatment method is considered as having a beneficial or therapeutic effect if contact, administration or delivery in vivo results in the use of a lesser amount, frequency or duration of another compound, agent, treatment or therapeutic regimen to treat the physiological condition, disorder, illness, disease, or symptom.

A sufficient amount or an effective amount can but need not be provided in a single administration and can but need not be administered alone (i.e., without a second drug, agent, treatment or therapeutic regimen), or in combination with another compound, agent, treatment or therapeutic regimen. In addition, a sufficient amount or an effective amount need not be sufficient or effective if given in single or multiple doses without a second compound, treatment, agent, or therapeutic regimen, since additional doses, amounts, frequency or duration of administration above and beyond such doses, or additional compounds, agents, treatments or therapeutic regimens may be included in order to be effective or sufficient in a given subject.

A sufficient amount or an effective amount need not be effective in each and every subject, nor a majority of subjects in a given group or population. Thus, a sufficient amount or an effective amount means sufficiency or effectiveness in a particular subject, not a group or the general population. As is typical for such methods, some subjects will exhibit a greater or less response to embodiments of the methods than other subjects.

As is typical for treatment or therapy, different subjects will exhibit different responses to treatment and some may not respond at all or may respond less than desired to a particular treatment protocol, regimen or process. Amounts effective or sufficient will therefore depend at least in part upon the disorder treated (e.g., the type or severity of the disease, disorder, illness, or symptom or pathology), the therapeutic effect desired, as well as the individual subject (e.g., the bioavailability within the subject, gender, age, etc.) and the subject's response to the treatment based upon genetic and epigenetic variability (e.g., pharmacogenomics).

Any compound, agent, treatment or other therapeutic regimen having a desired, beneficial, additive, synergistic or complementary activity or effect can be formulated or used in a combination with or in addition to embodiments of the methods. Methods embodiments therefore include additional treatments, protocols and therapies, which include any other composition, treatment, protocol or therapeutic regimen. In various aspects, the compound, agent, treatment or therapeutic regimen is for providing a subject with protection against, treatment of, decreasing susceptibility towards, treating an associated disorder caused by or associated with the physiological condition, disorder, illness, disease, or a symptom or pathology thereof.

Thus, composition and in vitro, ex vivo and in vivo methods embodiments can be combined with other agents or treatments or a method step. In various embodiments, an agent or treatment includes an anti-inflammatory agent or treatment or an immunosuppressive agent or treatment.

Anti-inflammatory agents useful in methods embodiments include cytokines and chemokines. Particular non-limiting examples of cytokines include anti-inflammatory cytokines such as IL-4 and IL-10. Anti-cytokines and anti-chemokines, such as antibodies that bind to pro-inflammatory cytokines, TNFα, IFNγ, IL-1, IL-2, IL-5, IL-6, IL-9, IL-13, IL-16, growth factors such as granulocyte/macrophage colony-stimulating factor can be employed, etc. Additional non-limiting examples of agents useful for treating inflammation include antibodies, such as anti-IgE (e.g., rhuMAb-E25 omalizumab), -IgA and -IgG antibodies, receptors and receptor ligands.

Additional non-limiting examples of agents or treatments to include in methods embodiments include immunosuppressive agents such as corticosteroids (steroid receptor agonists) such as budesonide, prednisone, flunisolide, flunisolide hydrofluroalkane, estrogen, progesterone, dexamethasone and loteprednol; beta-agonists (e.g., short or long-acting) such as bambuterol, formoterol, salmeterol, albuterol; anticholinergics such as ipratropium bromide, oxitropium bromide, cromolyn and calcium-channel blocking agents; antihistamines such as terfenadine, astemizole, hydroxyzine, chlorpheniramine, tripelennamine, cetirizine, desloratadine, mizolastine, fexofenadine, olopatadine hydrochloride, norastemizole, levocetirizine, levocabastine, azelastine, ebastine and loratadine; antileukotrienes (e.g., anti-cysteinyl leukotrienes (CysLTs)) such as oxatomide, montelukast, zafirlukast and zileuton; phosphodiesterase inhibitors (e.g., PDE4 subtype) such as ibudilast, cilomilast, BAY 19-8004, theophylline (e.g., sustained-release) and other xanthine derivatives (e.g., doxofylline); thromboxane antagonists such as seratrodast, ozagrel hydrochloride and ramatroban; prostaglandin antagonists such as COX-1 and COX-2 inhibitors (e.g., celecoxib and rofecoxib), aspirin; and potassium channel openers.

The terms “subject” and “patient” are used interchangeably herein and refer to animals, typically mammals, such as humans, non-human primates (gorilla, chimpanzee, orangutan, macaque, gibbon), domestic animals (dog and cat), farm and ranch animals (chickens, ducks, horses, cows, goats, sheep, pigs), experimental animals (mouse, rat, rabbit, guinea pig), laboratory and experimental animal (mouse, rat, rabbit, guinea pig) and humans. Animal models include, for example, disease model animals (e.g., such as mice, rats, rabbits, guinea pigs and non-human primates) for studying in vivo efficacy. Particular non-limiting examples include a mouse colitis model (see, e.g., Example 9), a mouse model of autoimmunity (BXSB) for lupus, experimental autoimmune encephalomyelitis (EAE) for multiple sclerosis, rheumatoid arthritis and inflammatory bowel disease, NOD mouse for insulin-dependent diabetes, collagen induced arthritis (CIA) for rheumatoid arthritis, etc., immunosuppression (Nude mice). Subjects include naturally occurring or non-naturally occurring mutated or non-human genetically engineered (e.g., transgenic or knockout) animals.

Subjects can be of any age. Human subjects include children, for example, newborns, infants, toddlers and teens, between the ages of 1 and 5, 5 and 10 and 10 and 18 years, adults between the ages of 18 and 60 years, and the elderly, for example, between the ages of 60 and 65, 65 and 70 and 70 and 100 years.

Subjects include mammals (e.g., humans) in need of treatment, that is, they may objectively or subjectively be likely to benefit from a treatment (e.g., a regulatory Tcell treatment). Such subjects include, for example, animals having an chronic or acute undesirable or aberrant immune response (e.g., a pathologic adaptive immune response), inflammation or inflammatory response (e.g., an autoimmune condition or disease). Subjects also include those at risk of having an acute or chronic undesirable or aberrant immune response (e.g., a pathologic adaptive immune response), inflammation or inflammatory response (e.g., an autoimmune condition or disease). Various benefits or improvements provided to a subject by various methods embodiments are as set forth herein or would be known to the skilled artisan for various physiological conditions, disorders, illnesses, diseases and symptoms and pathologies thereof.

Non-limiting candidate subjects include those having or at risk of having multiple sclerosis (MS), diabetes mellitus types I or II, rheumatoid arthritis (RA), juvenile rheumatoid arthritis, osteoarthritis, psoriatic arthritis, encephalomyelitis, myasthenia gravis, systemic lupus erythematosus (SLE), autoimmune thyroiditis, atopic dermatitis, eczematous dermatitis, psoriasis, Sjögren's Syndrome, intestinal inflammation, Crohn's disease, inflammatory bowel disease (IBD), ulcerative colitis, Celiac disease, aphthous ulcer, iritis, conjunctivitis, keratoconjunctivitis, asthma, allergic asthma, cutaneous lupus erythematosus, scleroderma, vaginitis, proctitis, erythema nodosum leprosum, auto immune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, polymyostitis, Wegener's granulomatosis, hepatitis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, immune-mediated infertility, autoimmune Addison's disease, pemphigus vulgaris, pemphigus foliaceus, dermatitis herpetiformis, autoimmune alopecia, Vitiligo, autoimmune hemolytic anemia, pernicious anemia, Guillain-Barre syndrome, Stiff-man syndrome, acute rheumatic fever, sympathetic ophthalmia, Goodpasture's syndrome, systemic necrotizing vasculitis, primary biliary cirrhosis and myelodysplastic syndrome.

Subjects further include those receiving or candidates for a cellular transplant (e.g., adult or embryonic stem cell, or bone marrow), tissue or organ transplant (e.g., liver, kidney, heart, lung, vein or artery, cornea), or graft (e.g., skin or muscle). Such subjects can exhibit an undesirable or aberrant immune response that leads to destruction of a transplanted cell(s), tissue or organ, as in transplant rejection or in graft vs. host disease (GVHD). Treating such a subject in accordance with a method embodiment can result in decreasing, reducing, inhibiting, suppressing, delaying, halting, limiting, controlling, abrogating, eliminating, blocking or preventing damage to or rejection of transplanted cell, tissue or organ or GVHD.

“At risk” subjects include those having risk factors associated with undesirable or aberrant immune response, inflammation or an inflammatory response, due to risk factors. Risk factors include gender, lifestyle (diet, smoking), occupation, environmental factors (allergen exposure), family history (autoimmune disease or disorders, MS, diabetes, etc.), genetic predisposition, etc. At risk subjects can therefore be identified by lifestyle, occupation, environmental factors, family history, and genetic screens. Susceptibility to autoimmune disease is frequently associated with MHC genotype. For example, in diabetes there is an association with HLA-DR3 and HLA-DR4.

Embodiments include pharmaceutical compositions or formulations, which are useful for administration, in vivo delivery or contact. Pharmaceutical compositions and formulations include carriers or excipients for administration to a subject.

As used herein the terms “pharmaceutically acceptable” and “physiologically acceptable” mean a biologically compatible formulation, gaseous, liquid or solid, or mixture thereof, which is suitable for one or more routes of administration, in vivo delivery or contact. A formulation is compatible in that it does not destroy activity of an active ingredient therein, or induce adverse side effects that far outweigh any prophylactic or therapeutic effect or benefit.

Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

The formulations may, for convenience, be prepared or provided as a unit dosage form. Preparation techniques include bringing into association the active ingredient and a pharmaceutical carrier(s) or excipient(s). In general, formulations are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient expression or activity, such as an inhibitor (e.g., antagonist), or an activator (e.g., agonist)) in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary active compounds (e.g., preservatives, antioxidants, antimicrobial agents including biocides and biostats such as antibacterial, antiviral and antifungal agents) can also be incorporated into the compositions. Preservatives and other additives include, for example, antimicrobials, anti-oxidants, chelating agents and inert gases (e.g., nitrogen). Pharmaceutical compositions may therefore include preservatives, antimicrobial agents, anti-oxidants, chelating agents and inert gases.

Preservatives can be used to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include, antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanide, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include those set forth above and, nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, valcyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Exemplary antifungals include agents such as benzoic acid, undecylenic alkanolamide, ciclopiroxolamine, polyenes, imidazoles, allylamine, thiocarbamates, amphotericin B, butylparaben, clindamycin, econazole, amrolfine, butenafine, naftifine, terbinafine, ketoconazole, elubiol, econazole, econazole, itraconazole, isoconazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, fluconazole, flutrimazole, nystatin, pimaricin, amphotericin B, flucytosine, natamycin, tolnaftate, mafenide, dapsone, caspofungin, actofunicone, griseofulvin, potassium iodide, Gentian Violet, ciclopirox, ciclopirox olamine, haloprogin, ketoconazole, undecylenate, silver sulfadiazine, undecylenic acid, undecylenic alkanolamide and Carbol-Fuchsin.

Pharmaceutical compositions can optionally be formulated to be compatible with a particular route of administration. Thus, pharmaceutical compositions include carriers (excipients, diluents, vehicles or filling agents) suitable for administration by various routes and delivery, locally, regionally or systemically, ex vivo or in vivo.

Exemplary routes of administration for contact or ex vivo or in vivo delivery include inhalation, respiration, intubation, intrapulmonary instillation, oral (buccal, sublingual, mucosal), intrapulmonary, rectal, vaginal, intrauterine, intradermal, topical, dermal, parenteral (e.g., subcutaneous, intramuscular, intravenous, intradermal, intraocular, intratracheal and epidural), intranasal, intrathecal, intraarticular, intracavity, transdermal, iontophoretic, ophthalmic, optical (e.g., corneal), intraglandular, intraorgan, intralymphatic.

Formulations suitable for parenteral administration include aqueous and non-aqueous solutions, suspensions or emulsions of the compound, which may include suspending agents and thickening agents, which preparations are typically sterile and can be isotonic with the blood of the intended recipient. Non-limiting illustrative examples of aqueous carriers include water, saline (sodium chloride solution), dextrose (e.g., Ringer's dextrose), lactated Ringer's, fructose, ethanol, animal, vegetable or synthetic oils. Examples of non-aqueous solvents are propylene glycol, polyethylene glycol, vegetable oils such as olive oil, and injectable organic esters such as ethyl oleate. Intravenous vehicles include fluid and nutrient replenishers, electrolyte replenishers (such as those based on Ringer's dextrose).

For transmucosal administration, penetrants can be included in the pharmaceutical composition. Penetrants are known in the art, and include, for example, for transmucosal administration, detergents, bile salts, and fusidic acid derivatives.

Pharmaceutical formulations and delivery systems appropriate for the compositions and methods of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

Embodiments including pharmaceutical formulations can be packaged in unit dosage forms for ease of administration and uniformity of dosage. A “unit dosage form” as used herein refers to a physically discrete unit suited as unitary dosages for treatment or administration; each unit containing a predetermined quantity of compound optionally in association with a pharmaceutical carrier (excipient, diluent, vehicle or filling agent) which, when administered in one or more doses, is calculated to produce a desired effect (e.g., a desired effect or benefit). Unit dosage forms can contain a daily dose or unit, daily sub-dose, or an appropriate fraction thereof, of an administered compound (e.g., an agonist) or cell (e.g., regulatory T cells). Unit dosage forms also include, for example, capsules, troches, cachets, lozenges, tablets, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. The individual unit dosage forms can be included in multi-dose kits or containers. Pharmaceutical formulations can be packaged in single or multiple unit dosage forms for ease of administration and uniformity of dosage.

Methods embodiments include contact or administration in vitro, ex vivo and in vivo at any frequency as a single bolus or multiple dose e.g., one, two, three, four, five, or more times hourly, daily, weekly, monthly or annually or between about 1 to 10 days, weeks, months, or for as long as appropriate. Exemplary frequencies are typically from 1-7 times, 1-5 times, 1-3 times, 2-times or once, daily, weekly or monthly. Timing of contact, administration ex vivo or in vivo delivery can be dictated by the physiological condition, disorder, illness, disease, or symptom or pathology thereof to be treated. For example, an amount can be administered to the subject substantially contemporaneously with, or within about 1-60 minutes, hours or days of the onset of a symptom or pathology of a chronic or acute undesirable, aberrant or pathologic immune response (e.g., adaptive) such as inflammation or an autoimmune disorder, or transplant rejection.

Methods include contact or administration in vitro, ex vivo or in vivo. Methods embodiments may be practiced via systemic, regional or local administration, by any route. Methods embodiments include administration to affected cells, or to an affected tissue or organ. In particular aspects, administration is to a skeletal joint or to gastro-intestinal tract.

A subject may be administered a single dose (e.g., bolus) or multiple doses (e.g., in divided/metered doses), which can be adjusted to be more or less according to the various considerations set forth herein and known in the art. Doses may vary depending upon the physiological condition, disorder, illness, disease or symptom to be treated, the onset, progression, severity, frequency, duration, probability of or susceptibility of the physiological condition, disorder, illness, disease or symptom to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome or beneficial effect desired, status of the physiological condition, disorder, illness, disease or symptom, any adverse side effects of the treatment or therapy, etc. For example, once control or a particular endpoint is achieved, for example, dose amount, frequency or duration can be reduced. The skilled artisan will appreciate the factors that may influence the dosage, frequency and timing required to provide an amount sufficient or effective for treatment.

For therapeutic treatment, a method is performed as soon as practical, typically within 0-72 hours or days after a subject manifests the physiological condition, disorder, illness, disease or a symptom or pathology thereof. For prophylactic treatment, a method is performed immediately or within 0-72 hours or days, or 0-4 weeks, e.g., 1-3 days or weeks, prior to anticipated or possible manifestation of the physiological condition, disorder, illness, disease or a symptom or pathology.

Doses can be based upon current existing treatment protocols, or amounts that are within or close to, but outside of, a physiological range. For example, retinoic acid, a retinoid X receptor (RXR) or a peroxisome proliferator activated receptor-gamma (PPARgamma) agonist can be administered to be in an amount in the subject at or near physiological (e.g., in serum) amounts (e.g., retinoic acid). In particular embodiments, the amount of a retinoic acid receptor agonist or retinoid X receptor (RXR) or peroxisome proliferator activated receptor-gamma (PPARgamma) agonist is approximately equivalent to physiological amounts of retinoic acid. In additional particular embodiments, the amount of a retinoic acid receptor agonist, retinoid X receptor (RXR) or peroxisome proliferator activated receptor-gamma (PPARgamma) agonist administered is such that the amount in the subject is about 1×10⁻⁹ M to about 5×10⁻⁵ M. In further particular embodiments, the amount of a retinoic acid receptor agonist, retinoid X receptor (RXR) agonist or peroxisome proliferator activated receptor-gamma (PPARgamma) agonist administered is such that the amount in the subject is less than about 1×10⁻⁹ M.

Doses can also be empirically, for example, using animal disease models or optionally in human clinical studies. Initial study doses can be based upon animal studies, such as primates, and the amount of compound administered to achieve a prophylactic or therapeutic effect or benefit.

The dose can be adjusted according to the mass of a subject, and will generally be in a range from about 25-250, 250-500, 500-1000, 1000-2500 or 2500-5000, 5000-25,000, 5000-50,000, 50,000-100,000 pg/kg; from about 0.1-1 ug/kg, 1-10 ug/kg, 10-25 ug/kg, 25-50 ug/kg, 50-100 ug/kg, 100-500 ug/kg, 500-1,000 ug/kg, 1-5 mg/kg, 5-10 mg/kg, 10-20 mg/kg, 20-50 mg/kg, 50-100 mg/kg, 100-250 mg/kg, 250-500 mg/kg, or more, of subject body weight, two, three, four, or more times per hour, day, week, month or annually. Of course, doses can be more or less, as appropriate, for example, 0.00001 mg/kg of subject body weight to about 10,000.0 mg/kg of subject body weight, about 0.001 mg/kg, to about 100 mg/kg, about 0.01 mg/kg, to about 10 mg/kg, or about 0.1 mg/kg, to about 1 mg/kg of subject body weight over a given time period, e.g., 1, 2, 3, 4, 5 or more hours, days, weeks, months, years. Exemplary dose amounts of retinoic acid receptor agonist, retinoid X receptor (RXR) agonist or peroxisome proliferator activated receptor-gamma (PPARgamma) agonist administered is in a range of from about 10 mg to 1200 mg, or from about 50 mg to 500 mg.

For a cell based method of treatment, doses can range from about 100,000 to about 1 billion cells. Exemplary dose amounts can be an amount of cells ranging from about 500,000 to about 500 million cells, or between about 1-100 million cells, or between about 1-10 million cells.

The invention provides, among other things, kits including regulatory T cells, cultures of regulatory T cells, dendritic cells, cultures of dendritic cells, combination compositions thereof and pharmaceutical compositions/formulations thereof, packaged into a suitable packaging material. In one embodiment, a kit includes packaging material, regulatory T cells, a culture of regulatory T cells, dendritic cells, or a culture of dendritic cells, and instructions. In various aspects, the instructions are for an in vitro, ex vivo or in vivo method, as set forth herein.

The term “packaging material” refers to a physical structure housing one or more components of the kit. The packaging material can maintain the components sterilely, and can be made of material commonly used for such purposes (e.g., paper, corrugated fiber, glass, plastic, foil, ampules, vials, tubes, etc.). A kit can contain a plurality of components, e.g., two or more regulatory T cell cultures.

A kit optionally includes a label or insert including a description of the components (type, amounts, doses, etc.), instructions for use in vitro, in vivo, or ex vivo, and any other components therein. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., floppy diskette, hard disk, ZIP disk), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacturer location and date, expiration dates.

Labels or inserts can include information for maintaining viability of cells, or information on a physiological condition, disorder, illness, disease or symptom, for which a kit component may be used. Labels or inserts can include instructions for a clinician or subject for using one or more of the kit components in an in vitro, ex vivo or in vivo method (e.g., treatment), as set forth herein. Instructions can include amounts, frequency or duration of administration, and instructions for carrying out any of the methods, treatment protocols or prophylactic or therapeutic regimes described herein.

Labels or inserts can also include information on any desired effect or benefit, or adverse side effects, a kit component may provide, such as a prophylactic or therapeutic effect or benefit. For example, a label or insert could provide a description of decreasing, reducing, inhibiting, suppressing, delaying, halting, limiting, controlling, abrogating, eliminating, blocking or preventing onset, severity, duration, progression, frequency or probability of the physiological condition, disorder, illness, disease or a symptom or pathology thereof.

Labels or inserts can further include information on potential adverse side effects. Labels or inserts can further include warnings to the clinician or subject regarding situations or conditions where a subject should stop or reduce use of a particular kit component. Adverse side effects could also occur when the subject has, will be or is currently taking one or more other medications that may be incompatible with treatment, or the subject has, will be or is currently undergoing another treatment protocol or therapeutic regimen which would be incompatible with treatment and, therefore, labels or inserts could include information regarding such side effects or incompatibilities.

Invention kits can moreover include a buffering agent, or a preservative or a stabilizing agent in a pharmaceutical formulation containing a compound of the invention. Each component of the kit can be enclosed within an individual container and all of the various containers can be within a single package. Invention kits can be designed for cold storage.

Invention kits can additionally include components, such as devices for practicing a method of the invention or administering regulatory T cells or dendritic cells, to a subject, ex vivo or in viva. The device can be a delivery device, such as a syringe, an IV bag or bottle, or an extracorporeal or implantable device.

Unless otherwise defined, all technical and scientific terms used herein have the same meaning as commonly understood by one of ordinary skill in the art to which this invention relates. Although methods and materials similar or equivalent to those described herein can be used in the practice or testing of the present invention, suitable methods and materials are described herein.

All publications, patents and other references cited herein are incorporated by reference in their entirety. In case of conflict, the present specification, including definitions, will control.

As used herein, the singular forms “a”, “and,” and “the” include plural referents unless the context clearly indicates otherwise. Thus, for example, reference to “a regulatory T cell” or a “regulatory T cell culture” includes a plurality of regulatory T cells and cultures; and reference to “a symptom” or “pathology” includes a plurality of symptoms or pathologies (e.g., adverse or undesirable). Of course, this does not preclude limiting certain embodiments of the invention to particular symptoms or pathologies, particular conditions, disorders, diseases or illnesses, particular subjects, treatment methodology, etc., using appropriate language.

The invention is generally disclosed herein using affirmative language to describe the numerous embodiments. The invention also includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless expressly or inherently disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This Example includes a description of various materials and methods.

### Mice

C57BL/6 CD45.1 (Ly5.1) and CD45.2 (Ly5.2), OT-I TCR-transgenic (C57BL/6 background), RAG1^{-/-} (C57BL/6 background), B7.1/2 double-knockout (C57BL/6 background) and IL-2^{-/-} (C57BL/6 background) mice were purchased from The Jackson Laboratories (USA). To obtain B7.1/2-IL-2 tripleknockout mice, B7.1/2^{-/-} and IL-2^{-/-} mice were crossed in our animal facility. OT-II CD90.1 TCR transgenic mice (C57BL/6 background). Mice were maintained under specific pathogen-free conditions and sentinel mice from the RAG1^{-/-}mice colony were tested to be negative for Helicobacter spp. and Citrobacter rodentium. Mice were used at 7-12 weeks of age. Animal care and experimentation were consistent with the NIH guidelines and were approved by the Institutional Animal Care and Use Committee at the La Jolla Institute for Allergy and Immunology.

### Antibodies

The following mouse antibodies were purchased from BD-Biosciences (USA), as conjugated to FITC, PE, PE-Cy5, PerCP-Cy5.5 or APC: α⁴β⁷ (LPAM-1), CD4 (L3T4), CD8 (53-6.7), CD11b(MI/70), CD11c (HL3), CD25 (PC61), CD45R (RA3-6B2), CD45RB (16A), CD45.1 or Ly5.1 (A20), CD45.2 or Ly5.2 (104), IL-17, IFN-γ (XMG1.2), NK1.1 (PK136), TCRα2 (B20.1), TCRβ5 (MR9-4), TCRβ chain (H57-597), TER-119 and isotype controls. Anti-mouse IL-2 (JES6-1A12), CD103 or α^{E}β⁷(2E7), CCR9 (CW199), CTLA-4 (UC10) and Foxp3 (FJK-16S) were purchased from eBioscience (USA). Anti-mouse CD16/32 used for Fc receptor blocking was isolated and purified in our laboratory.

### T Cell, Antigen Presenting Cell (APC) and Dendritic Cell (DC) Isolation

For CD4⁺CD25⁻ T cell isolation, spleens were removed, teased into cell-single suspensions and filtered through a 70 μm cell strainer (Fisher Scientific, USA). CD4⁺CD25⁻_{_{T}} cells were isolated by negative selection after incubation with a mix of specific biotin-conjugated mAbs (anti-CD8, CD11b, CD11ε, CD25, CD45R (B220), NK1.1, and TER119) at 1/200 dilution, and anti-biotin magnetic microbeads (25-40 μl per spleen) (Miltenyi Biotec, USA).

For CD8⁺T cell isolation, spleens cells were removed and isolated using a CD8 T cell isolation kit, according the manufacturer's protocol (Miltenyi Biotec, USA). APCs were isolated from spleen of C57BL/6 mice by negative selection using CD90.2 (Thy1.2) magnetic microbeads to deplete T cells (Miltenyi Biotec). Red blood cells (RBC) in the splenic cell suspension were removed using a RBC lysing buffer (Sigma, USA) and cells were irradiated with 3000 Rads.

For dendritic cells isolation, spleen and MLN were chopped and digested by collagenase type D (Roche, USA) for 30 min at room temperature. Digested tissues were treated with 5 mM of EDTA for additional 5 min and mashed through a 70 μm cell strainer. Subsequently, CD11c⁺ cells were enriched by positive selection using anti-CD11c microbeads according to the manufacturer's protocol (Miltenyi Biotec, USA). Enriched populations were stained with CD45-APC, CD 11c-PE, I-A^{b}-FITC and TCR β and sorted for TCRβ⁻CD45⁺CD11c⁺I-Ab⁺ by flow cytometry using a FACS-DIVA cell sorter (USA).

### In Vitro T Cell Stimulation

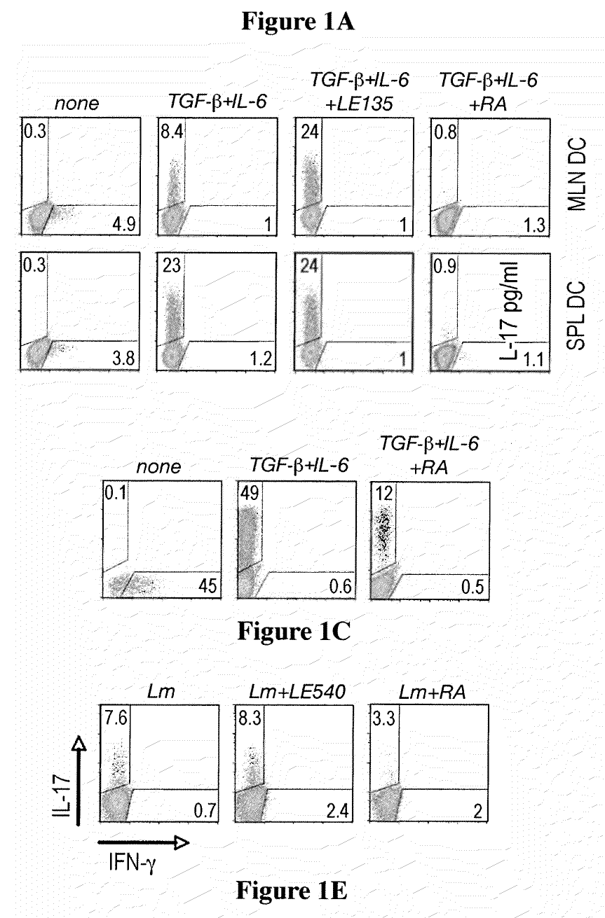

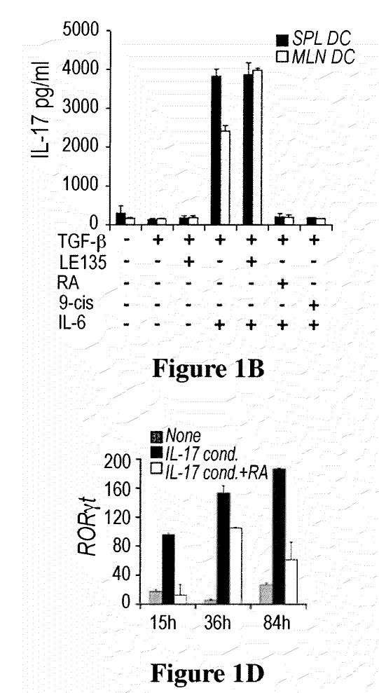

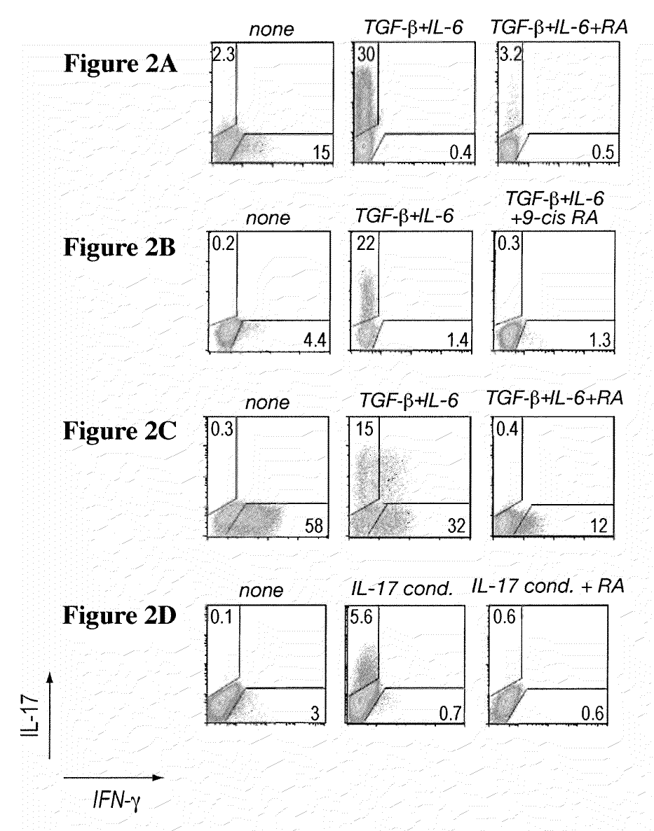

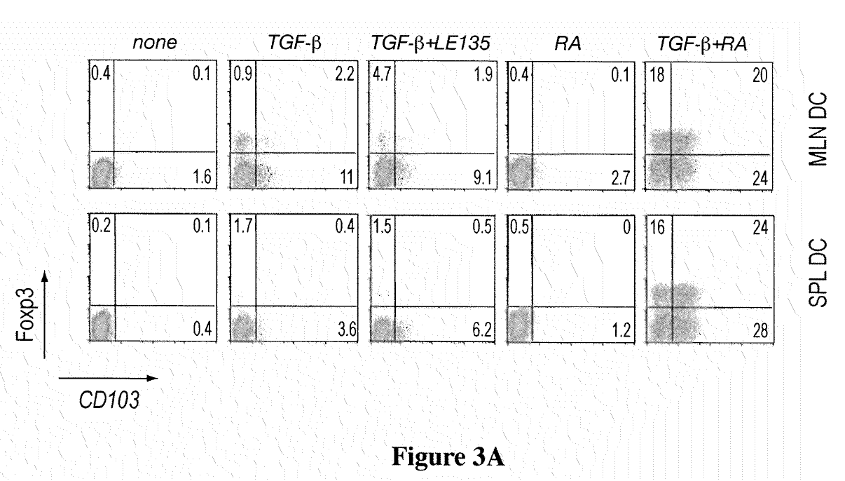

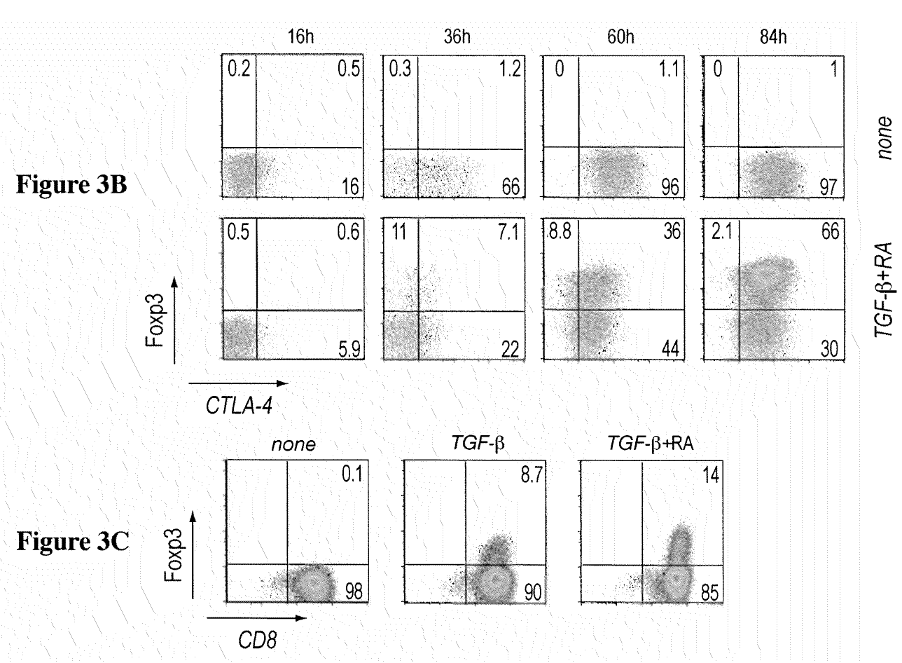

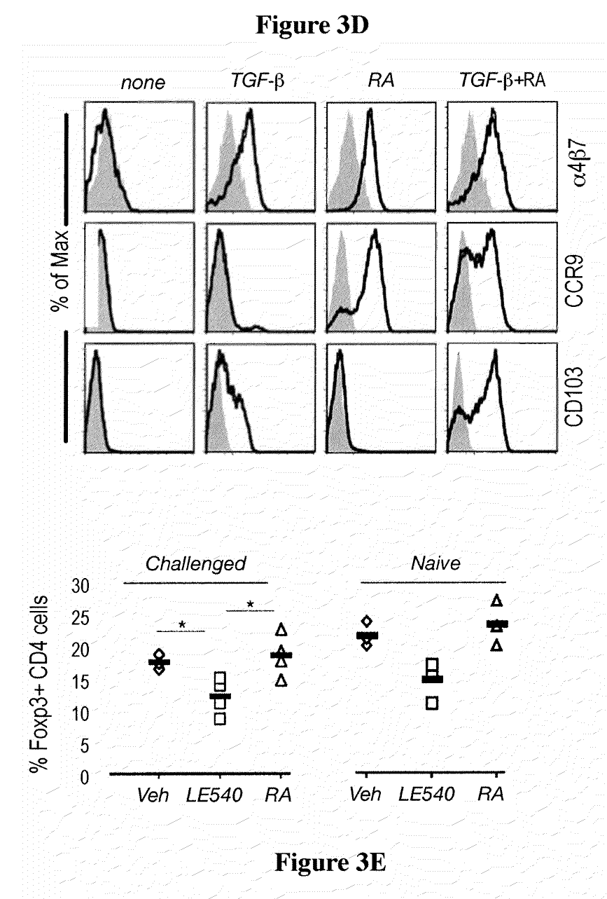

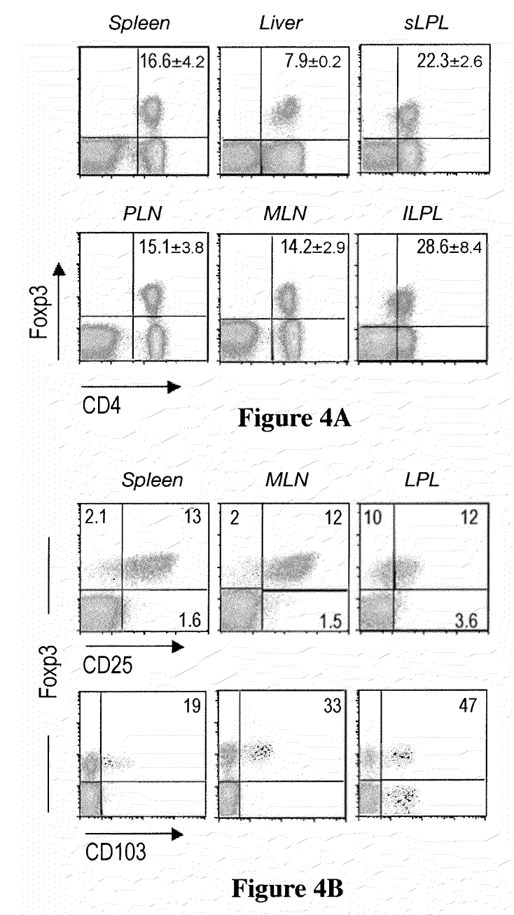

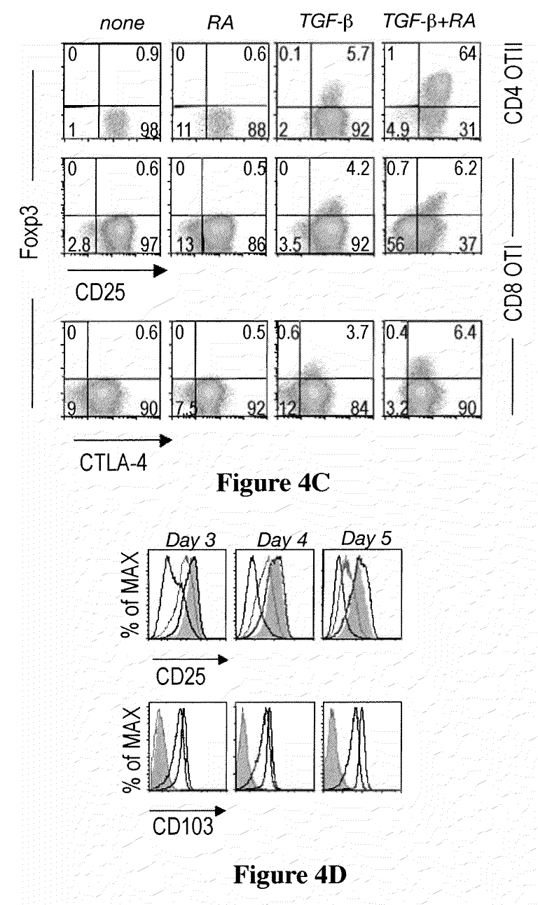

The culture medium used for dendritic cell-T cell cultures was Iscove's modified Dulbecco medium (IMDM) (Gibco, USA) supplemented with 10% heat-inactivated FCS, 2 mM L-glutamine, 100 U/ml penicillin, 100 μg/ml streptomycin, and 5 mM β-mercaptoethanol (all Sigma, USA). In the remaining cultures, RMPI (Gibco, USA) supplemented in the same way as IMDM was used. For dendritic cell-T cell cultures, 2×10⁴ dendritic cells and 1×10⁵ T cells were cultured in a volume of 200 μl (96-well plates). For irradiated APC-T cell cultures, 5×10⁵ APCs and 1×10⁵ T cells were cultured in a volume of 200 μl in 96-well plates (2×10⁶ APCs and 0.5×10⁶ T cells per well in 48-well plates). Polyclonal CD4 or CD8 T cells were stimulated with 1 μg/ml of soluble anti-CD3ε (45-2C11), immobilized anti-CD3ε (5 μg/ml) and 2 μg/ml anti-CD28 (BD-Biosciences, USA) (FIG. 1D), or mouse CD3/CD28 T cell expander beads according to the manufacturer's protocol (Invitrogen, USA) (FIG. 2D). For the study shown in FIG. 4A, after sorting of naive CD4⁺ cells (CD25⁻CD44^{low}CD62L⁺), cells were washed twice in PBS+0.1% BSA, counted and labeled with 2 μM of CFSE (Carboxyfluorescein diacetate, succinimidyl ester) (Molecular Probes, USA) at a concentration of 5−10×10⁶ cells per ml in PBS-0.1% BSA. For the study described in FIGS. 3D and E, after three and one half days in culture stimulated as described above, cells were washed, rested for two days with rmIL-2 (100 u/ml), washed and re-stimulated with 1 μg/ml of soluble anti-CD3a and specified cytokines for additional three days before analysis.

Exogenous cytokines used were IL-2 (200 u/ml), IL-6 (20 ng/ml), TGF-β1 (5 ng/ml), TGF-β2 or TGF-β3, at 5 ng/ml (R&D Systems, USA), IL-1β (10 ng/ml) and TNF-α (10 ng/ml) (Peprotech Inc., USA). MHC-I (OVA²⁵⁷⁻²⁶⁴) and MHC-II (OVA³²³⁻³³⁹)-restricted OVA peptides recognized by OT-I and OT-II TCR-transgenic T cells, respectively, were synthesized by Agent Inc. (USA) and used at 1 nM (OT-I OVAp) or 1 μM (OT-II OVAp).

All-trans retinoic acid (RA) and 9-cis retinoic acid (9-cis RA) were purchased from Sigma (USA), dissolved in DMSO (10 mM) and stored at −20° C. and protected from light and used at a 100 nM concentration. Retinoid acid receptor antagonist LE135 was dissolved in ethanol (10 mM) and added to cultures at 1 μM concentration. Retinoid acid receptor antagonist LE540 (Wako, Japan) was dissolved in DMSO (1 mM) and added to cultures at 1 μM concentration.

Listeria monocytogenes Infection

Listeria monocytogenes (strain WT LMOVA) was used. Mice were orally infected (gavage) with 5×10⁸ CFU of L. monocytogenes. Mice received i.p. injections of vehicle, RA or LE540 on the same day and 2 days after infection. Mice were sacrificed 5 days post-infection for analysis.

All-trans retinoic acid (RA) was purchased from Sigma (USA), dissolved in 1:1 DMSO+soybean oil (3 mg/ml, for i.p. injections) or soybean oil alone (6 mg/ml, for gavage), stored at −20° C. and protected from light and used at 300 μg/mouse. Retinoid acid receptor antagonist LE540 (Wako, Japan) was dissolved in 1:1 DMSO+soybean oil (0.5 mg/ml), stored at −20° C. and protected from light and used at 100 μg/mouse. Every second day, in a interval of two weeks, naïve mice received gavage of either vehicle, RA or LE540.

### Experimental Colitis Model

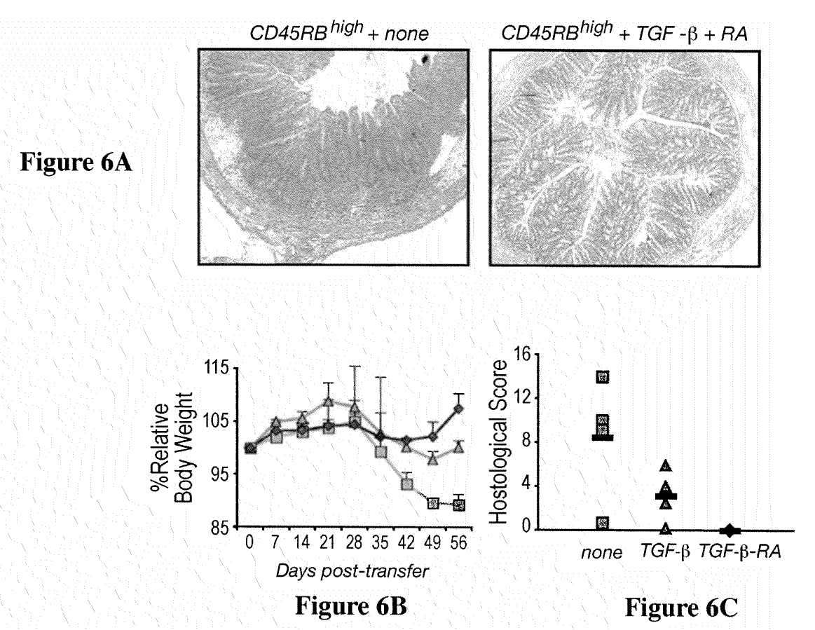

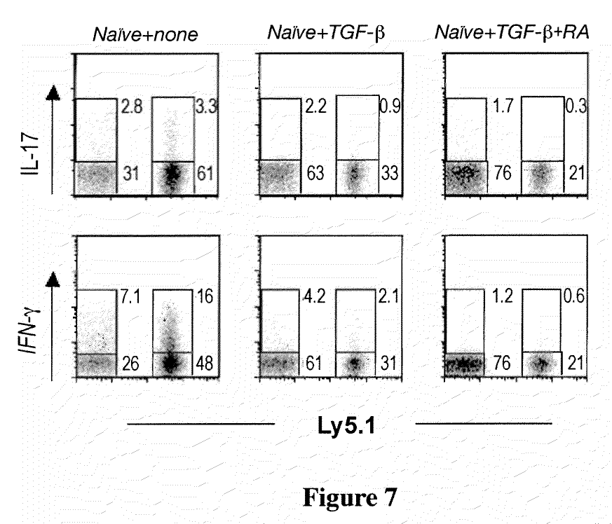

Spleens were removed from the donor mice (C57BL/6) and teased into cell-single suspensions in HBSS media (Invitrogen, USA). Cell suspensions were filtered through a 70 μm cell strainer and subsequently, CD4⁺ cells were enriched by positive selection using anti-CD4 (L3T4) magnetic microbeads according to the manufacturer's protocol (Miltenyi Biotec, USA). The CD4⁺-enriched cells were washed twice and stained with PE-Cy5-conjugated anti-CD4 and PE-conjugated anti-CD45RB antibodies. After staining, cells were washed and the CD4⁺CD45RB^{high} T cell population was sorted by flow cytometry using a FACS-DIVA cell sorter. Purified CD4⁺CD45RB^{high} naïve T cells were washed twice, resuspended in PBS and injected into RAG^{-/-} recipient mice. Recipients were injected intravenously with 5×10⁵ cells in 200 μl of PBS. For co-transfer studies (FIGS. 6 and 7), in vitro conditioning of CD4 T cells was performed using 48-well-plates containing 0.5×10⁶ sorted CD4⁺CD45RB^{high} cells from C57BL/6 Ly5.1 mice and 2×10⁶ irradiated splenic APCs from C57BL/6 mice. These cells were stimulated as described above and, after 3.5 days, CD4⁺T cells were isolated using CD4 magnetic microbeads. RAG^{-/-} recipients were injected intravenously with 5×10⁵ freshly isolated CD4⁺CD45RB^{high} cells from C57BL/6 Ly5.1 and 2.5×10⁵ in vitro-conditioned CD4⁺T cells in 200 μl of PBS. Transferred mice were monitored regularly for signs of disease including weight loss, hunched over appearance, pilo-erection of the coat, and diarrhea.

At the corresponding time points following transfer, diseased animals were sacrificed for histological analysis. Tissue samples of 3-5 mm obtained from distal and proximal portion of the large intestine were fixed in 4% formalin. Fixed tissue was later embedded in paraffin and 3 μm sections were prepared and stained with hematoxylin-eosin. To evaluate the severity of the inflammation samples were coded and scored by a pathologist in a blinded fashion using a previously described scoring system [Aranda et al., J. Immunol 158, 3464 (1997)]. Scores were averaged to represent the severity of disease. Higher scores (maximum 14) indicate greater pathology.

### Preparation of Liver, Intraepithelial and Lamina Propria Lymphocytes

Liver lymphocytes were isolated using 37.5% isotonic Percoll (GE Healthcare, USA), as previously described [Y. Kinjo et al, Nat Immunol 7, 978 (2006)]. Intestinal lymphocytes were isolated and prepared as previously described [Aranda et al., J Immunol 158, 3464 (1997)]. Briefly, small and large intestines were removed and placed in chilled HBSS media containing 5% FCS. The intestines were carefully cleaned from the mesentery and flushed of fecal content. Intestines were opened longitudinally and then cut into 1 cm pieces. The intestinal tissue was transferred to a 250-ml Erlenmeyer flasks containing 25 ml of preheated HBSS complemented with 2% FCS and 1 mM DTT (Sigma, USA) and shaken at 200 rpm for 40 min at 37° C. The tissue suspension was passed through a stainless steel sieve into 50-ml conical tubes and the cells were pelleted by centrifugation at 1200 rpm for 10 min at 4° C. The cell pellet was resuspended in complete HBSS, layered over a discontinuous 40/70% Percoll gradient, and centrifuged at 2000 rpm for 30 min. Cells from the 40/70% interface were collected, washed and resuspended in complete RPMI media. These purified cells constituted the intraepithelial lymphocyte (IEL) population. To isolate the lamina propria lymphocytes (LPL), the remaining intestinal tissue in the stainless steel sieve was minced and transferred to conical tubes. The minced pieces were resuspended in 20 ml of complete RPMI containing 1 mg/ml of collagenase (Sigma, USA) and shaken at 200 rpm for 40 min at 37° C. The tissue suspension was collected and passed through a 70 μm cell strainer and the cells were pelleted by centrifugation at 1200 rpm. The cells were then resuspended and layered onto a 40/70% Percoll gradient, centrifugated and processed as described above for the IEL preparation.

### Flow Cytometry Analysis and Intracellular Cytokine Staining

Spleen, peripheral lymph node (PLN) and MLN were removed and then mashed through a 70 μm cell strainer and RBC in the cell suspension were removed using a RBC lysing buffer. Liver mononuclear cells, IEL and LPL were isolated as described above. Prior to staining, cells were washed and resuspended in staining buffer containing 1×PBS, 2% BSA, 10 mM EDTA and 0.01% NaN3. To block non-specific staining, the 2.4G2 anti-CD16/32 antibody was added. Antibodies for cell surface markers were added and cells were incubated 25 min on ice. Following the staining, the cells were washed twice and analyzed the same day or fixed in PBS containing 1% paraformaldehyde and 0.01% NaN3 and analyzed later in a FACS-Calibur (BD-Bioscience, USA). For intracellular cytokine staining, cells obtained from in vitro cultures or isolated IELs were incubated for 4-5 hours with 50 ng/ml PMA, 750 ng/ml Ionomycin (both Sigma, USA) and 10 μg/ml Brefeldin A (Invitrogen, USA) in a tissue culture incubator at 37° C. Surface staining was performed for 25 min with the corresponding cocktail of fluorescently labeled antibodies. After surface staining, cells were resuspended in Fixation/Permeabilization solution (BD Cytofix/Cytoperm kit-BD PharMingen, USA), and intracellular cytokine staining was performed according to the manufacturer's protocol.

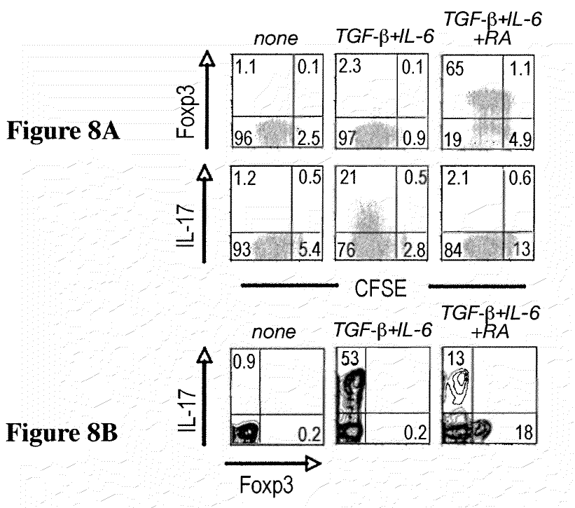

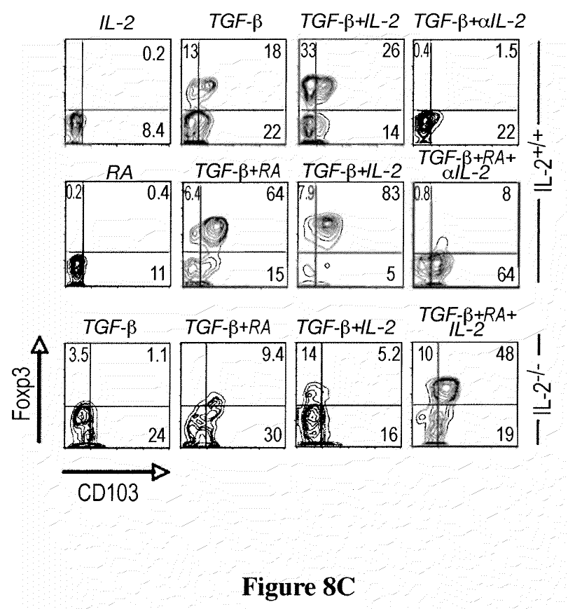

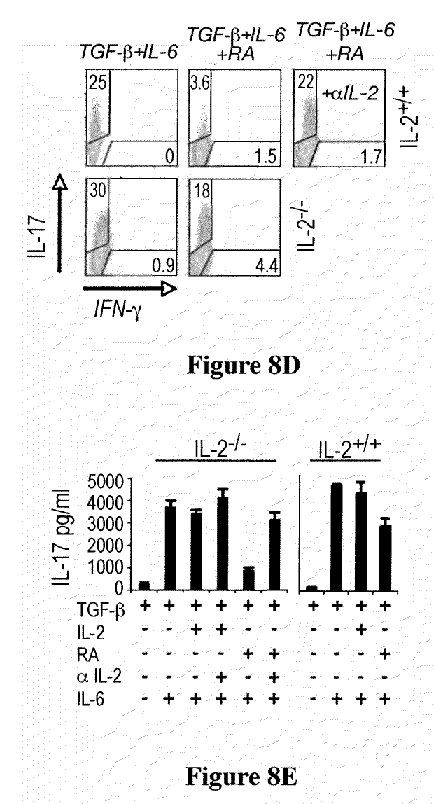

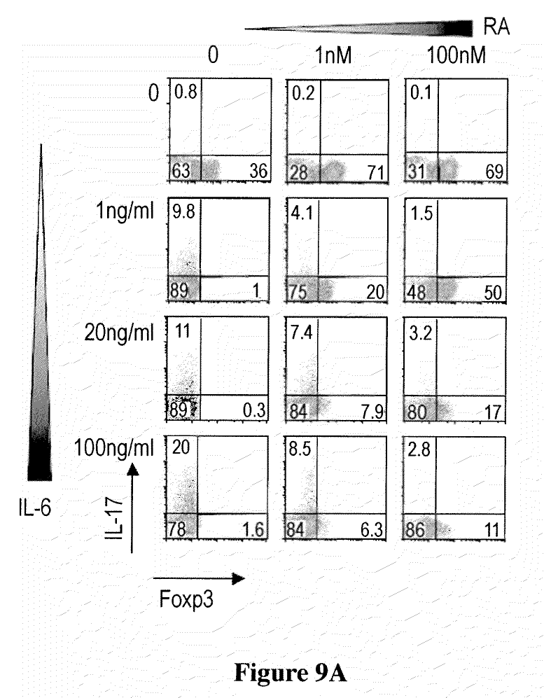

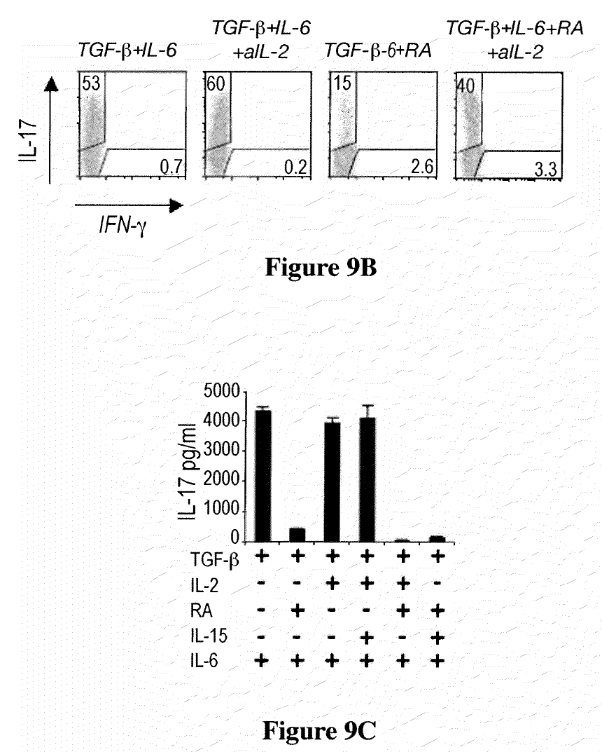

For Foxp3 staining, no stimulation with PMA/Ionomycin was performed, with the exceptions of FIGS. 8B and 9A. In these cases, the time of incubation with PMA/Ion was reduced to 3.5 hours. Intracellular Foxp3 staining was performed as per the eBioscience's Foxp3-staining kit protocol.

### Detection of Cytokines by ELISA

After in vitro stimulation of cells, IFN-γ and IL-17 in the culture supernatants were quantified by ELISA. Capture and detection antibodies for IFN-γ and IL-17 and recombinant cytokines standards for IFN-γ and IL-17 ELISAs were purchased from BD-Biosciences (USA).

### RNA Isolation and Real-Time RT-PCR

For analysis of RORγ-T mRNA expression, naïve CD4⁺T cells were purified essentially as described by Ivanov et al. [Ivanov, II et al., Cell 126, 1121 (2006)]. In brief, CD4+ T cells were purified from spleens of C57BL/6 mice using anti-CD4 magnetic microbeads (Miltenyi Biotec) and MACS columns. CD4⁺T cells were stained with anti-CD25-PE, anti-CD4-PECy5, anti-CD62L-FITC, and anti-CD44-APC. The CD4⁺CD25⁻ CD44^{low}CD62L⁺ T cells population was sorted by flow cytometry using a FACS-DIVA cell sorter (>99% purity). Some studies were also performed using CD4⁺CD45RB^{high} T cell population, sorted as described above.

At different time-points, cells were harvested, washed with sterile PBS and resuspended in TRIZol (Invitrogen, USA). Samples were then frozen and kept at −80° C. for later utilization. For RNA isolation, the whole tissue was homogenized and RNA was separated from DNA and proteins by precipitation with chloroform and extraction with isopropanol. The cDNA was synthesized from the total RNA using the Superscript II system (Invitrogen, USA) following the instructions provided by the manufacturer. Subsequently, the cDNA was subject to real-time PCR using SYBR green (Bio-Rad Laboratories, USA) and the following mouse primers: RORγt forward: 5′-CCGCTGAGAGGGCTTCAC-3′, RORγt reverse: 5′-TGCAGGAGTAGGCCACATTACA-3′, L32 forward, 5′-GAAACTGGCGGAAACCCA-3′ and L32 reverse, 5′GGATCTGGCCCTTGAACCTT-3′. Gene expression was normalized to L32. Data were collected and analyzed on an iCycler Bio-Rad (Hercules, USA).

### Example 2

This Example includes studies comparing the ability of gut-associated dendritic cells and peripheral dendritic cells in driving Th-17 differentiation.

IL-17 and IFN-γ staining of gated TCR Vβ5⁺CD4⁺ spleen cells from OT-II TCR transgenic mice was performed, as shown in FIG. 1A. CD4⁺CD25⁻ cells were stimulated with OVAp and MLN or spleen (SPL) dendritic cells, and where indicated, with exogenous cytokines and LE135 or all-trans retinoic acid (RA). Intracellular staining of gated TCRβ⁺CD4⁺ cells for IL-17 and IFN-γ of polyclonal CD4⁺CD25⁻ spleen T cells stimulated with soluble α-CD3 was performed, with irradiated spleen cells, and with added cytokines and RA as indicated in FIG. 2A. Mesenteric lymph node (MLN) dendritic cells and spleen dendritic cells were used to stimulate OVA peptide (OVAp) specific, OT-II TCR transgenic CD4 T cells (FIG. 1A), or α-CD3 stimulated polyclonal CD4 T cells (FIG. 2A). In the presence of IL-6 and TGF-β, MLN dendritic cells displayed reduced capacity to induce Th-17 differentiation as compared to their splenic counterparts.

### Example 3

This example includes studies indicating that RA suppresses differentiation of Th-17 cells.

To study if the reduced capacity of MLN dendritic cells to drive Th-17 differentiation might also be controlled by RA, the RA receptor (RAR) antagonist, LE135 [Hashimoto, et. al., J Biol Chem 274, 15360 (1999)] was included during in vitro activation of T cells under conditions that promote Th-17 differentiation.

In addition to the studies represented in FIGS. 1A and 2A (discussed above), intracellular staining of gated TCRβ⁺CD4⁺ cells for IL-17 and IFN-γ of with OT-II TCR⁺CD4⁺CD25⁻ spleen T cells stimulated with the relevant OVAp, sorted spleen CD11c+ dendritic cells and with added cytokines and 9-cis RA (100 nM) as indicated was also performed, gated on TCR Vβ5⁺CD4⁺ cells (FIG. 2B). In these studies, the relative inefficiency of MLN dendritic cells to mediate Th-17 differentiation was reversed, such that they primed T cells at levels similar to spleen dendritic cells. By comparison, addition of all-trans RA to the cultures inhibited the Th-17 differentiation by spleen dendritic cells (FIGS. 1A, 1B, and in FIG. 2A) and it was found that the addition of vitamin-A metabolite, 9-cis RA, to the cultures also inhibited the Th-17 differentiation by spleen dendritic cells (FIG. 2B).

Intracellular staining of gated TCRβ⁺CD4⁺ cells for IL-17 and IFN-γ of OT-I TCR⁺CD8⁺T cells stimulated with the relevant OVAp and spleen CD11c⁺ dendritic cells and without (none) or with the indicated cytokines, without or with RA, was performed (FIG. 2C). Intracellular staining of gated TCRβ⁺CD4⁺ cells for IL-17 and IFN-γ of polyclonal CD4⁺CD25⁻ spleen T cells stimulated with anti-CD3/CD28 beads and without exogenous cytokines (none) or with the indicated cytokines (IL-17 cond.: TGF-β, IL-6, IL-1β, TNF-α) and with or without RA, was also performed, gated on TCRβ+CD4+ cells (FIG. 2D).

As shown in FIG. 1C, intracellular IL-17 and IFN-γ staining of gated TCRβ⁺CD8⁺ cells was performed. Total CD8⁺spleen T cells were stimulated with α-CD3ε and spleen APCs with the indicated cytokines, (none, TGF-β⁺IL-6, and, TGF-β⁺ IL-6 and RA). In addition to CD4⁺Th-17 cells, it was observed that WT (FIG. 1C) or OT-I TCR⁺ (FIG. 2C discussed above) cytotoxic CD8⁺T lymphocytes activated in the presence of TGF-β and IL-6 also generated IL-17⁺T cells, and that RA was again able to inhibit this. This study indicated that T cells can become IL-17⁺ cells regardless of their effector phenotype and that RA specifically suppresses IL-17 expression.

Th-17 cells can also be generated in the absence of dendritic cells if additional cytokines, such as IL-1 and TNF-α, are included in the culture conditions. Under such APC-free conditions, RA also inhibited the generation of IL-17⁺T cells, demonstrating that RA targets T cells directly (discussed above in FIG. 2D) RORγt is an orphan nuclear receptor that has been implicated in the gene transcription of Th-17 cells [Ivanov et al., Cell 126, 1121 (2006)]. To determine if RA controls RORγt, CD4 T cells were activated under Th-17 culture conditions, with or without RA.

As shown in FIG. 1D, RORγt mRNA was analyzed at various times by PCR in CD4 T cells stimulated with α-CD3ε and α-CD28. Where indicated, IL-17 inducing cytokines (IL-17 cond. shown in black), or these cytokines plus RA (shown in white) were included (mean±SD). The results indicate that the presence of inflammatory cytokines, TGF-β induced high levels of RORγt, whereas added RA greatly reduced its expression (FIG. 1D).

### In Vivo Studies

To study the apparent suppressive effect of RA on Th-17 development in vivo, mice were orally infected with Listeria monocytogenes (Lm) and treated with RA or an RAR inhibitor (LE540). As shown in FIG. 1E intracellular IL-17 and IFN-γ staining of CD4⁺T cells from small intestine lamina propria was performed 5 days after oral infection with Listeria monocytogenes. Data are representative of 3-4 mice per group. A measurable reduction of Th-17 mucosal T cells was seen in the animals that received RA, while RAR inhibitor-treated mice showed no apparent difference compared to the controls (FIG. 1E). Collectively, these studies suggest that RA acts to oppose Th-17 development in vitro and in vivo, and that this appears to operate directly on T cells via the reduction of RORγt.

### Example 4

This example includes studies indicating that RA regulates the reciprocal differentiation of TGF-β-dependent Treg and Th-17 cells.

The inefficiency of MLN dendritic cells to promote Th-17 differentiation, and the reciprocal TGF-β dependent conversion to either Th-17 cells or Tregs, led to studies to determine if mucosal dendritic cells might drive enhanced TGF-β-dependent Treg differentiation. TGF-β-dependent Tregs were reported to be identified by expression of the forkhead-winged helix transcription factor, Foxp3 [(Fontenot, et al. Nat Immunol 4, 330 (2003); Hori, et al, Science 299, 1057 (2003); Khattri, et al., Nat Immunol 4, 337 (2003); Mucida et al., J Clin Invest 115, 1923 (2005)], Foxp3 induction in OT-II CD4 cells cultured with spleen or MLN dendritic cells in the presence of TGF-β and OVAp was studied. As shown in FIG. 3A intracellular staining for Foxp3 and surface CD103 of gated TCR Vβ5⁺CD4⁺ cells from OT-II TCR transgenic mice, was performed. Additionally, CD4⁺CD25⁻T cells were stimulated with OVAp and MLN or SPL dendritic cells, and as indicated, with TGF-β1 and LE135 or RA. In contrast to Th-17 differentiation seen with splenic dendritic cells, MLN dendritic cells were able to induce higher frequencies of Foxp3⁺ cells (FIG. 3A) In the presence of RAR inhibitor, TGF-β-dependent Foxp3 induction by MLN dendritic cells was reduced, but enhanced by the addition of RA (FIG. 3A).

Additionally, the frequency of Foxp3⁺CD4 T cells in total CD4 T cells isolated from different tissues was analyzed. As shown in FIG. 4A intracellular staining of Foxp3 and CD4 expression by TCRβ⁺ gated T cells isolated from various tissues was performed; sLPL and ILPL indicate small and large intestine lamina propria lymphocytes, respectively, and PLN indicates peripheral lymph node. The numbers represent mean±SEM of the percentage of Foxp3⁺ T cells in the CD4⁺T cell population. As shown in FIG. 4B, intracellular Foxp3 staining and surface staining was performed for CD25 or CD103 of gated TCRβ⁺ CD4⁺ T cells; in the lower panels, the numbers indicate the percentage of CD103⁺ cells in the Foxp3⁺ population. Five mice were analyzed for each study. This analysis indicated that the small and large intestine CD4⁺ lamina propria lymphocytes (LPLs) consistently harbored more Foxp3⁺ cells as compared to other CD4⁺T cell subsets (FIG. 4A). Regardless of the tissue, most Foxp3⁺ CD4 cells also expressed the IL-2 receptor (IL-2R) α chain, CD25 (FIG. 4B), whereas a larger proportion of the Foxp3⁺ CD25⁺CD4⁺ cells in the intestinal tissues also coexpressed CD103, the αE integrin subunit expressed by many mucosal T cells (FIG. 3B). The enhanced frequency of Foxp3⁺ cells in LP T lymphocytes suggested that priming by mucosal dendritic cells might favor the peripheral differentiation of Foxp3⁺ Tregs.

In addition to the expression of Foxp3, peripherally generated Tregs also induce CTLA-4 as part of their functional differentiation. To examine if the synergistic effect of exogenous RA and TGF-β also controls the expression of CTLA-4, CTLA-4 expression was analyzed at different time-points during the in vitro culture of naïve OT-II CD4 T cells stimulated in the presence of RA and/or TGF-β. To study this, intracellular Foxp3 and CTLA-4 staining of OT-II TCR CD4⁺CD25⁻T cells stimulated as in FIG. 3A (discussed above) was performed, except with spleen APCs instead of dendritic cells (FIG. 3B).

As indicated in FIG. 3B, induction of CTLA-4 on Foxp3⁻ and Foxp3⁺ activated cells was delayed in the presence of TGF-β and RA (FIG. 3B). At later time-points, the majority of cells cultured with TGF-β and RA were both CTLA-4 and Foxp3⁺ (FIG. 3B). These studies indicate that the synergistic effect of TGF-β and RA inhibits the early CTLA-4 expression on effector cells, but instead allows later expression of CTLA-4 by cells committed to the Foxp3 lineage.

As discussed, RA promoted the expression of CTLA-4, a cell surface receptor typically expressed by Tregs, on most TGF-β-generated Foxp3⁺ cells (FIG. 3B). CD8⁺T cells from OT-I TCR transgenic mice were stimulated with OVAp and spleen dendritic cells with TGF-β1 and RA and intracellular staining of gated TCRβ⁺ cells for Foxp3 was performed (FIG. 3C). Intracellular staining for Foxp3 and CTLA-4 and surface staining for CD25 of OT-I TCR⁺CD8⁺T cells stimulated with the relevant OVAp and irradiated spleen APCs for 3 days and without (none) or with the indicated cytokines, and without or with RA was performed (FIG. 4C). For comparison, OT-II CD4⁺CD25⁻ cells stimulated under the same conditions are also shown in FIG. 4C. Histograms representing staining of the OT-I CD8⁺ cells, gated on TCRβ⁺CD8⁺ cells, stimulated as described above, in the Brief Description of the Drawings (FIG. 4D) for 3, 4 and 5 days. Solid grey—none; grey line—RA; dashed line—TGF-β; black line—TGF-b+RA. The synergistic effect of RA on TGF-b-dependent Foxp3⁺ T cell differentiation was also apparent with CD8⁺T cells (FIG. 3C), indicating that the RA-mediated increase of Foxp3⁺ T cell differentiation might not be limited to CD25⁺CD4⁺Tregs, (FIGS. 4C and 4D). These studies indicate that RA treated CD8 T cells, but not CD4 T cells, down-regulate CD25 expression upon in vitro activation in the presence of TGF-β and RA.

On the other hand, TGF-β and RA synergized to upregulate CD103 expression on CD8 cells, similarly to what was described for CD4 T cells. The down-regulation of CD25 occurs almost exclusively on the Foxp3 negative cells (the majority of activated CD8 T cells). The Foxp3⁺ CD8 cells (polyclonal or TCR-transgenic), however, express high levels of CD25⁺ and CTLA-4. It is possible that the CD25⁻ CD8 T cells become dependent on IL-15 instead of IL-2, similarly to the IL-15 dependency of CD8⁺ IEL in vivo. Overall, these data indicate that RA controls the reciprocal differentiation of TGF-β dependent Treg and Th-17 cells.

### Example 5

This example includes studies showing that the combination of RA and TGF-β results in cell populations having different homing capacities.

Mucosal dendritic cell-derived RA has also been reported to mediate the induction of gut homing receptors, including the integrin α₄β₇ and CCR9, specific for homing to the small intestine as reported in [Iwata et al., Immunity 21, 527 (2004)], whereas TGF-β has been reported to promote the induction of CD103, the α_{E} subunit of the α_{E}β₇ integrin [Hadley, J Immunol 159, 3748 (1997)]. Studies were performed to determine whether TGF-β and RA might synergize to induce these receptors.

Cell surface staining of gated TCRβ⁺CD4⁺ cells for CD103, α₄β₇ and CCR9 was performed. CD4⁺CD25⁻T cells were stimulated with soluble α-CD3ε and spleen APCs plus TGF-β1, RA, or TGF-β1 and RA. Isotype controls are indicated with solid gray histograms, and representative data from three studies are shown in FIG. 3D. Consistent with synergy, RA greatly enhanced TGF-β-mediated CD103 expression, in contrast however, TGF-β partially antagonized RA-induced CCR9 (FIG. 3D). These results show that the combination of RA and TGF-β results in CCR9+Tregs with tropism for the small intestine and CCR9⁻ Foxp3⁺ cells with different homing capacity.

### Example 6

This Example includes data from studies to determine the influence of RA in vivo.

Listeria monocytogenes (Lm) infected mice were treated with RA or the RAR inhibitor. FIG. 3E shows the percentage of Foxp3⁺CD4⁺ cells in CD4⁺ TCRβ⁺ lymphocytes from the small intestine lamina propria 5 days after oral infection with Listeria monocytogenes (left panel) or in naïve controls (right panels). * P<0.05 (test T-student). Although RA alone was not found to measurably enhance the differentiation of Foxp3⁺Tregs in vivo, inhibition of RAR did significantly reduce the number of mucosal Foxp3⁺Treg cells in Lm challenged mice (FIG. 3E)

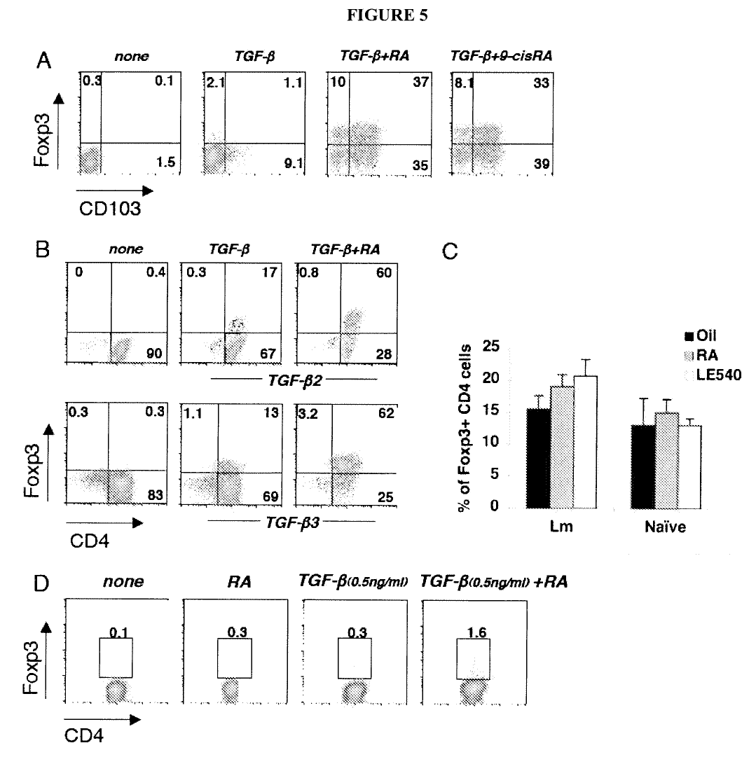

FIG. 5C shows the percentage of Foxp3⁺CD4⁺ lymphocytes in total CD4⁺ TCRβ⁺ T cells isolated from the spleen of mice 5 days after oral infection with Listeria monocytogenes. Each group received 2 i.p. injections (days 0 and 2) with vehicle, RA or LE540. Data of naïve mice that received 2-week of gavage treatment with vehicle, RA or LE540 are shown on the right side (mean±SD). No effect of RA or RAR on spleen Foxp3⁺CD4 cells was observed as indicated in (FIG. 5C). The finding that RA combined with TGF-β but not alone, can drive differentiation of Foxp3⁺T cells in vitro (FIG. 2A) indicates that TGF-β might be a limiting factor in the lack of Treg differentiation induced by exogenous RA in vivo (FIG. 5D).

### Example 7

This example includes studies indicating that Retinoic acid enhances TGF-β-mediated Foxp3 induction.

Intracellular staining was performed as follows: (FIG. 5A) for Foxp3 and CD103 of OT-II TCR⁺CD4⁺CD25⁻ spleen T cells stimulated with the relevant OVAp, sorted spleen CD11c⁺ dendritic cells and without exogenous cytokines (none) or with indicated cytokines, and without or with RA or 9-cis RA (both at 100 nM); gated on TCR Vβ5⁺CD4⁺ cells; (FIG. 5B) Intracellular staining of Foxp3 and CD4 staining of naïve polyclonal CD4⁺CD25⁻ spleen T cells stimulated with soluble α-CD3ε, irradiated spleen cells and without exogenous cytokines (none) or with TGF-β2 or TGF-β3 and without or with RA. This data indicated that the other relevant Vitamin-A derivative, 9-cis RA, had a similar increasing effect on in vitro generated Foxp3+Tregs (FIG. 5A) and RA synergized equally with TGF-β1, TGF-β2 or TGF-β3 isoforms to induce enhanced Foxp3⁺T cell differentiation (FIG. 5B).

Additionally, intracellular staining for Foxp3 of OT-II CD4 T cells stimulated in the same conditions as described for FIG. 5A was performed and the representative data are shown in FIG. 5D. Under low amounts of TGF-β (0.5 ng/ml, or 10 times less than the normal concentration), RA still enhances TGF-β-dependent Foxp3 induction in TCR-transgenic CD4 cells, however, overall Foxp3 induction is greatly reduced, confirming TGF-β as a critical and limiting factor in this Treg differentiation (5D).

### Example 8

This example includes co-transfer studies in which TGF-β plus RA in vitro differentiated Tregs regulate in vivo.

To examine if in vitro generated Foxp3⁺CD4 T cells could function to suppress effector T cells in vivo, co-transfer studies were performed using naïve CD45RB^{hi}CD4 T cells that induce colitis in immune deficient mice [Izcue, et al., Immunol Rev 212, 256 (2006)] in combination with CD4 T cells previously cultured under different conditions. As shown in FIGS. 6(A-E): (A) Hematoxylin and eosin staining of distal colon of RAG-1^{-/-} mice 6-7 weeks after co-transfer of 5×10⁵ CD4⁺CD45RB^{hi} cells with 2.5×10⁵ CD4⁺T cells stimulated in vitro with α-CD3ε alone (none) or with TGF-β1 and RA was performed with original magnification, 40× and representative data from 4 mice in each group. (B) Body weight of RAG-1^{-/-} mice after transfer of 5×10⁵ CD4⁺CD45RB^{hi}cells with 2.5×10⁵ α-CD3ε stimulated CD4⁺T cells with no additions (squares), TGF-β1 (triangles), or TGF-β1 and RA (diamonds). The mean±SD weight of four animals per group is shown; data are representative of three studies. (C) Histological scores of the groups described in (B). (D) Foxp3 intracellular staining of naïve TCRβ⁺CD4⁺ that were initially stimulated with soluble α-CD3ε and spleen APCs with the indicated cytokines. The cells were rested for two days with IL-2, and re-stimulated in the absence of exogenous cytokines before analysis. (E) Intracellular staining for IL-17 of naïve CD4⁺T cells initially stimulated and rested as described in (D), but in the presence of TGF-β and IL-6, and re-stimulated in the indicated conditions. Percentage of IL-17⁺ cells in the gated TCRβ⁺CD4⁺ cells is depicted.

Mice that received CD4 T cells activated without cytokine conditioning, combined with naïve CD45RB^{hi}CD4 cells, developed severe colitis (FIG. 6). In contrast, recipients that were co-transferred with CD4 T cells activated in the presence of TGF-β were partially protected from disease, and mice co-transferred with naïve T cells and CD4 T cells activated in vitro in the presence of both TGF-β and RA, showed no apparent signs of disease (FIG. 6).

Additionally, intracellular staining was performed for IL-17 and IFN-γ of IELs from large intestine isolated from RAG^{-/-}recipient mice, 6-7 weeks after transfer of 5×10⁵ Ly5.2⁺ (Ly 5.1−) CD4⁺CD45RB^{hi} cells together with 2.5×10⁵ CD4⁺ T cells (Ly5.1⁺) stimulated in vitro with α-CD3ε alone or together with TGF-β1 or TGF-β1 and RA. Gated on CD4⁺ lymphocytes (FIG. 7). Fewer mucosal CD4 T cells isolated from these animals produced IL-17 and IFN-γ (FIG. 7). These results suggest that Foxp3⁺T cells generated in vitro with RA and TGF-β have a measurable regulatory capacity and are able to control inflammation upon transfer in vivo. In addition, whereas Tregs generated in vitro by TGF-β alone lose Foxp3 expression upon re-stimulation, the majority of RA+TGF-β differentiated Foxp3⁺T cells remained Foxp3⁺ after re-stimulation, showing that RA drives differentiation of a stable Treg lineage (as shown in FIG. 6D). Conversely, RA and TGF-β also suppressed committed Th-17 cells in secondary cultures whereas TGF-β alone did not (FIG. 6E).

### Example 9

This example includes studies of the reciprocal TGF-β dependent T cell differentiation by IL-6 and RA.

To ascertain whether RA counteracts the activity of IL-6, TGF-β-dependent T cell differentiation in the presence of RA together with IL-6 was analyzed. FIG. 8A shows CFSE labeled naïve CD4⁺T cells were stimulated with α-CD3ε, spleen APCs, with the indicated cytokines and, as indicated, with RA. TNF-α, IL-1-β, TGF-β and IL-6, were used to drive IL-17 differentiation. Intracellular staining of gated TCRβ⁺CD4⁺ cells for Foxp3 and IL-17 is depicted. FIG. 8B shows intracellular staining for Foxp3 and IL-17 of CD8⁺T cells stimulated with soluble α-CD3ε and spleen APCs under the indicated conditions. CD4 (FIG. 8A) or CD8 (FIG. 8B) T cells cultured with RA under conditions that otherwise promote TGF-β-dependent Th-17 differentiation, converted to Foxp3⁺ cells with a decrease in Th-17 differentiation.

Additionally, intracellular staining for Foxp3 and IL-17 of polyclonal CD4⁺CD25⁻T cells stimulated with soluble α-CD3ε, irradiated spleen APCs and TGF-β (5 ng/ml) was performed, without or together with indicated concentrations of IL-6 and RA. Gated on TCRβ⁺CD4⁺ cells (FIG. 9A). The results indicate that the antagonistic effect of RA on IL-6 was dose dependent (FIG. 9A) indicating that under physiological conditions, the RA-driven TGF-β dependent Treg differentiation can override the IL-6 promoted TGF-β-dependent Th-17 generation.

### Example 10

This example includes studies of RA-mediated effects on T cell differentiation in vitro in the absence of IL-2.

IL-2 is required for the production of TGF-β dependent Foxp3⁺Treg cells [Davidson et al., J Immunol 178, 4022 (2007)]. Recently it was reported that exogenously added IL-2 also suppresses Th-17 differentiation [Laurence et al., Immunity 26, 371 (2007)]. To determine if RA-mediated regulation of T cell polarization required IL-2 signaling, RA-mediated effects on T cell differentiation in vitro were examined in the absence of IL-2, using anti-IL-2 or IL-2−/−T cells.

As shown in FIGS. 8C and 8D, the following was performed: intracellular staining for Foxp3 and surface staining for CD103 (C); or for intracellular IL-17 and IFN-γ (D) of naïve CD4⁺T cells from B7.1/2^{-/-} IL-2^{+/+} or B7.1/2^{-/-} L-2^{-/-} mice. Cells were stimulated with soluble α-CD3ε, spleen APCs and the indicated cytokines, RA and/or blocking antibody to IL-2 (20 μg/ml), gated on TCRβCD4⁺ cells. As shown in FIG. 8E ELISA for IL-17 in the supernatant of the cultures in (C) and (D) (mean±SD) was performed.

As shown in FIGS. 9B and 9C the following was also performed: (B) intracellular staining for IL-17 and IFN-γ of total CD8⁺ T cells from C57BL/6 mice stimulated with soluble α-CD3ε, irradiated spleen APCs and without (none) or with the indicated cytokines and/or RA (100 nM) and/or blocking anti-IL-2 antibodies (20 μg/ml); (C) ELISA for IL-17 in the supernatants of the cultures set up as described in FIG. 9B with the conditions indicated.

In these studies, the enhanced effect of RA to drive differentiation of Foxp3⁺ cells and the inhibitory effect of RA on TGF-β-dependent Th-17 differentiation, were for the most part dependent on IL-2 (FIGS. 8C, 8D, 8E and 9B and 9C). RA mediated regulation did not require exogenously added IL-2, although when added together RA and exogenous IL-2 synergized to drive the reciprocal regulation of TGF-β-dependent T cell differentiation (FIG. 8). However, RA- and exogenous IL-2-controlled differentiation appeared distinct, in that RA-mediated TGF-β-dependent Foxp3 differentiation generated mostly CD103⁺Tregs, whereas the majority of the IL-2-driven Foxp3⁺Tregs were CD103⁻ (FIG. 8C). Conversely, the mechanism of RA and IL-2 to suppress Th-17 cells also appeared to have some distinctions, since RA measurably decreased IL-17 cytokine secretion whereas exogenous IL-2 did not (FIG. 8E). Collectively, these studies show that although both exogenous IL-2 and RA require initial IL-2 signaling for their regulatory function, the cooperation with TGF-β and the further downstream mechanisms they activate may be very different.

The transcription factors STAT5 and STAT3/RORγt have been reported to be involved in transcription of Foxp3 and IL-17, respectively [(Ivanov, II et al., Cell 126, 1121 (2006); X. O. Yang et al., J Biol Chem (2007); A. Laurence et al., Immunity 26, 371 (2007)]. RORγt shows strong homology with RARs and both function in the context of transcriptional activators and repressors (Winoto, et al., Cell 109 (2002). Similar to STAT3/RORγt, STAT5 and RAR are also connected and they can physically interact and bind to overlapping DNA binding sites to promote coordinated transcription activity [(Si., et al., Blood 100, 4401 (2002)]. In addition RAR and STAT5 bind the same repressor, SMRT, which can be released by RA [(Nakajima et. al., Embo 20, 6836 (2001)]. Furthermore, RA might also synergize with Smads that act downstream of TGF-β receptor signaling, and/or with the transcription factor Runx3, which is involved in CD103 induction and physically interacts with Smads to cooperate in TGF-β mediated signaling [(Woolf, et. al., Dev Biol (2006)].

The reciprocal activity of RA in inhibiting TGF-β-dependent Th-17 generation, while promoting Foxp3⁺Treg differentiation might provide a self-correcting mechanism for TGF-β to regulate both pro- and anti-inflammatory immunity. This regulatory capacity has particular relevance for the intestine, where efficient immune protection has to coincide with maintaining the mucosal barrier integrity.

Vitamin A deficiency causes immune dysfunction and increased mortality [A. Sommer, J Infect Dis 167, 1003 (1993)]. This study has offered evidence that RA-mediated effects might be important in vivo. It is possible that the immune pathology characteristic of sub-physiological levels of RA might in part be attributed to an imbalanced TGF-β function favoring pro-inflammatory Th-17 cells at the expense of anti-inflammatory Tregs.

### Example 11

This example includes a study of RALDH isoform expression.

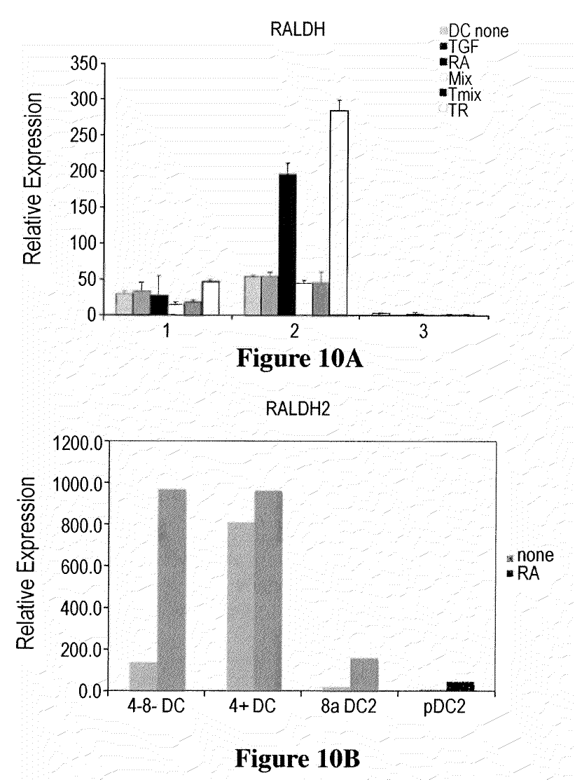

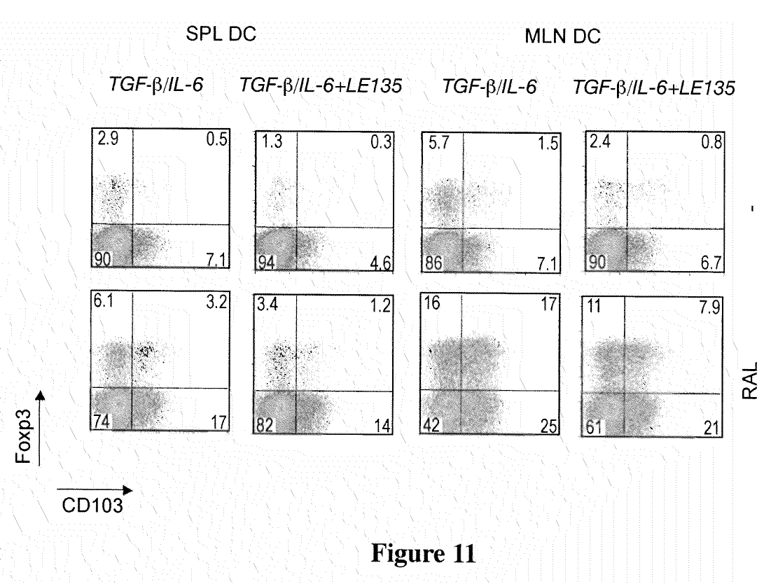

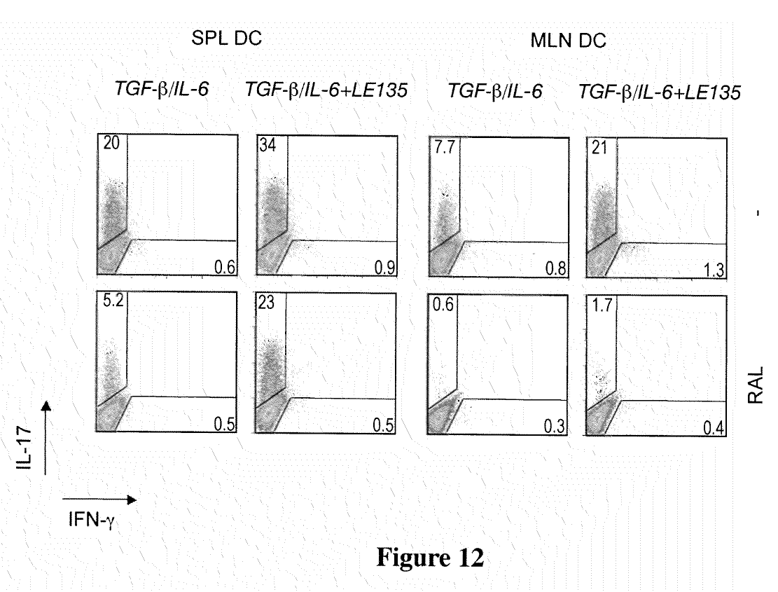

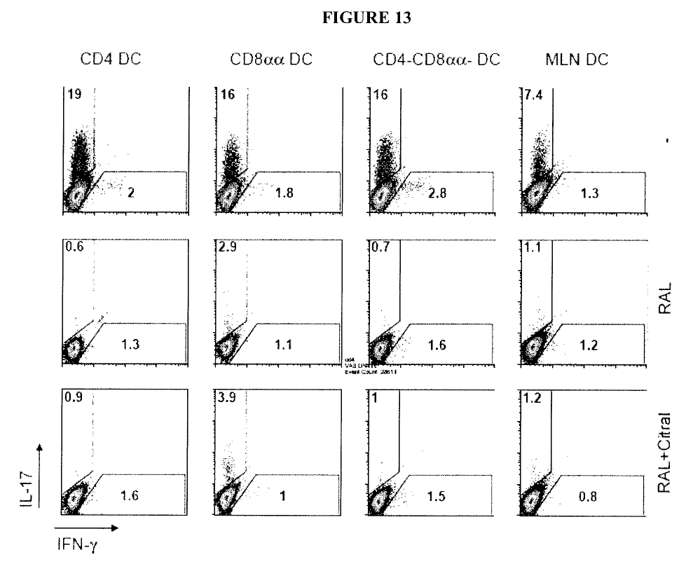

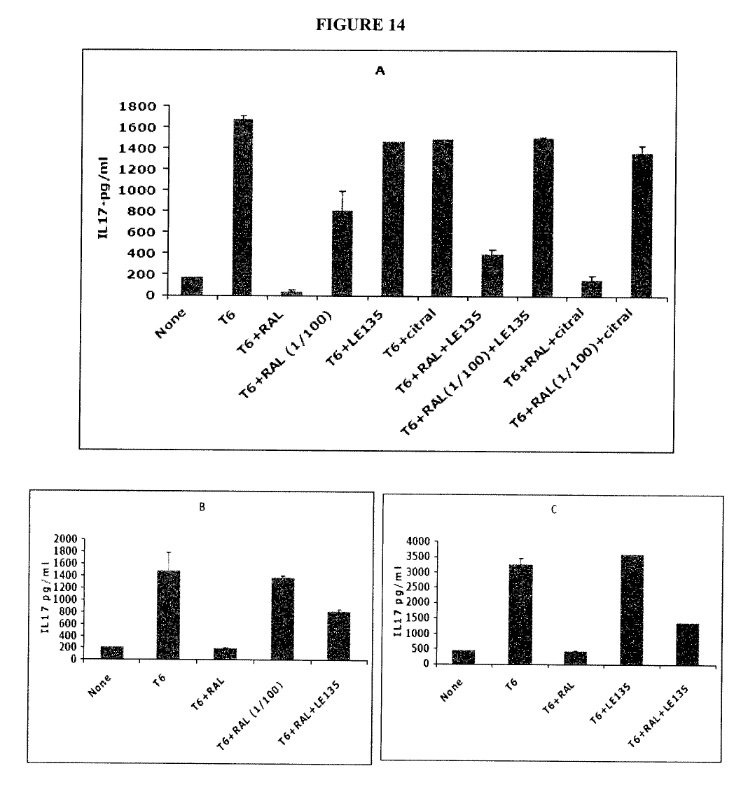

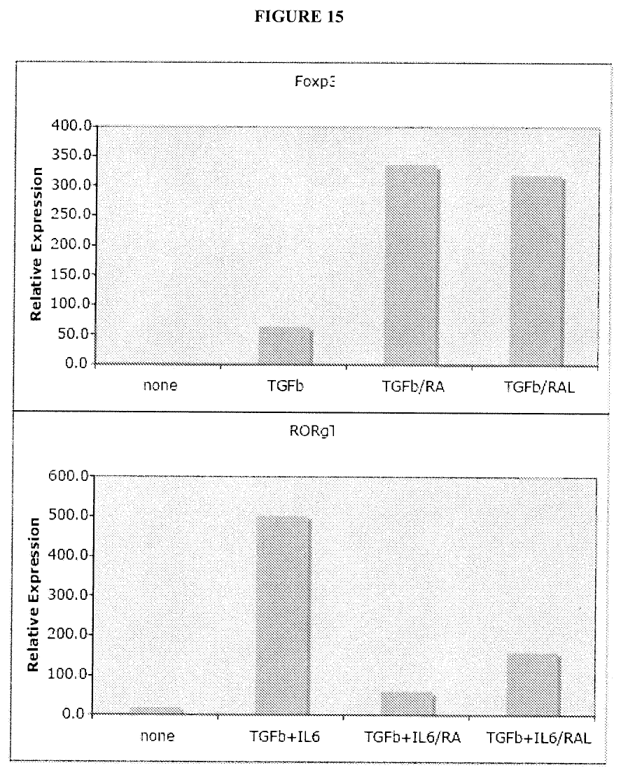

As shown in FIG. 10 expression of mRNA, was measured by qPCR, for the RALDH enzyme isoforms 1, 2 and 3 (10A) or only RALDH2 (10B) by sorted total splenic CD11c⁺dendritic cells (10A) or CD11c⁺dendritic cells sorted in subpopulations that express CD4, CD8 or plasmacytoid dendritic cells (10B). After sorting, dendritic cells were incubated for 18 h in the presence of only medium (none), different cytokines (TGF-β; mix=IL-1⁺IL-6⁺TNF-α; Tmix=TGF-β⁺ mix; TR=TGF-β+RA or retinoic acid (RA), mRNA was extracted and cDNA was generated.

1. A method of stimulating or increasing differentiation to regulatory T cells, comprising contacting blood cells or T cells with an amount of TGF-beta or TGF-beta analogue and a retinoic acid receptor agonist, or an amount of a retinoid X receptor (RXR) or peroxisome proliferator activated receptor-gamma (PPARgamma) agonist, sufficient to stimulate or increase differentiation to regulatory T cells.

2. The method of claim 1, wherein the retinoic acid receptor agonist is vitamin A, or a vitamin A derivative, analogue or metabolite.

3. The method of claim 2, wherein the vitamin A metabolite comprises retinoic acid, or a retinoic acid derivative, analogue or isomer.

4. The method of claim 2, wherein the retinoic acid derivative comprises an ester or an amide.

5. The method of claim 2, wherein the retinoic acid derivative comprises fenretinide or retinaldehyde.

6. The method of claim 2, wherein the retinoic acid analogue comprises 9-cis retinoic acid, 13-cis retinoic acid or all trans retinoic acid.

7. The method of claim 3, wherein the retinoic acid isomer comprises an arotinoid.

8. The method of claim 7, wherein the arotinoid comprises adapalene or tazarotene.

9. The method of claim 1, wherein the retinoid X receptor (RXR) or peroxisome proliferator activated receptor-gamma (PPARgamma) agonist is retinal, or a retinal derivative, stereoisomer, analogue or metabolite.

10. The method of claim 9, wherein the retinal derivative, stereoisomer, analogue or metabolite is an all-trans, 13-cis, 11-cis, 9-cis, 7-cis, 11,13-cis or 9,13-cis vitamin A aldehyde, or a hydrate, a hemiacetal or an acetal form.

11. The method of claim 9, wherein the retinal derivative, stereoisomer, analogue or metabolite is Retinal hydrate; Retinal methyl hemiacetal; Retinal ethyl hemiacetal; Retinal propyl hemiacetal; Retinal isopropyl hemiacetal; Retinal butyl hemiacetal; Retinal pentyl hemiacetal; Retinal octyl hemiacetal; Retinal benzyl hemiacetal; Retinal dimethyl acetal; Retinal diethyl acetal; Retinal dipropyl acetal; Retinal diisopropyl acetal; Retinal dibutyl acetal; Retinal dipentyl acetal; Retinal dioctyl acetal; Retinal dibenzyl acetal; Retinal propylene glycol hemiacetal or acetal; Retinal 1,2-O-isopropylidene glyceryl hemiacetal or acetal; Retinal 3-allyloxy-1,2-propanediol hemiacetal or acetal; Retinal phythyl hemiacetal; Retinal diphytyl acetal; Retinal dodecyl hemiacetal; or Retinal didodecyl acetal.

12. The method of claim 9, wherein the retinal derivative is 5,6-dioxo-5,6-seco-retinal, 5,6-dihydro-5,6-epoxy-retinal, or 4-oxoretinal.

13. The method of claim 1, wherein the blood cells comprise peripheral blood mononuclear cells (PBMC).

14. (canceled)

15. The method of claim 1, wherein the blood cells or the T cells are mammalian.

16. The method of claim 1, wherein the blood cells or the T cells are human.

17. The method of claim 1, wherein the blood cells or the T cells are contacted in vitro or in vivo.

18. The method of claim 1, further comprising proliferating or expanding the regulatory T cells in vitro, ex vivo or in vivo.

19. The method of claim 1, further comprising contacting the blood cells or the T cells with a TGF-beta agonist.

20. The method of claim 1, further comprising contacting the blood cells or the T cells with IL-2.

21. The method of claim 1, wherein the T cells comprise naïve T cells or activated T cells.

22. The method of claim 21, wherein the activated T cells comprise T cells characterized as exhibiting increased expression of CD44 and reduced expression of CD45, as compared to naïve T cells.

23-24. (canceled)

25. An in vitro culture of regulatory T cells that express a marker associated with regulatory T cells, wherein said regulatory T cells are in the culture in an amount greater than the amount of regulatory T cells in a culture after contact of blood cells with TGF-beta or a TGF-beta analogue without a retinoid X receptor (RXR) or peroxisome proliferator activated receptor-gamma (PPARgamma) agonist agonist, or wherein said regulatory T cells are in the culture in an amount greater than the amount of regulatory T cells in a culture after contact of blood cells with TGF-beta or a TGF-beta analogue without a retinoic acid receptor agonist.

26.-29. (canceled)

30. The regulatory T cells of claim 23 or the an in vitro culture of regulatory T cells of claim 25, wherein the marker associated with regulatory T cells comprises Foxp3, CCR9 and alpha4beta7.

31.-33. (canceled)

34. The regulatory T cells of claim 23 or the an in vitro culture of regulatory T cells of claim 25, wherein at least a portion of the regulatory T cells maintain expression of Foxp3, CD103, CCR9, alpha4beta7, CD25 or CTLA4 markers, survive or proliferate after introduction into or administration to a subject.

35.-41. (canceled)

42. A method of producing or increasing numbers of regulatory T cells, comprising contacting blood cells or T cells with a retinoic acid receptor agonist, or a retinoid X receptor (RXR) or peroxisome proliferator activated receptor-gamma (PPARgamma) agonist, in an amount that produces or increases numbers of regulatory T cells.

43. The method of claims 1 or 42, further comprising contacting cells with an antigen or an anti-CD3 antibody.

44. The method of claim 43, wherein the antigen is a self antigen.

45. A method of inhibiting or decreasing differentiation to activated or effector T cells, comprising contacting T cells with a retinoic acid receptor agonist, or a retinoid X receptor (RXR) or peroxisome proliferator activated receptor-gamma (PPARgamma) agonist, in an amount that inhibits or decreases differentiation to activated or effector T cells.

46. A method of reducing numbers of TH-17+ effector cells, comprising contacting TH-17+ effector cells with a retinoic acid receptor agonist, or a retinoid X receptor (RXR) or peroxisome proliferator activated receptor-gamma (PPARgamma) agonist, in an amount that reduces numbers of TH-17+ effector cells.

47. The method of claims 45 or 46, wherein the retinoic acid receptor agonist is vitamin A, or a vitamin A derivative, analogue or metabolite.

48. The method of claims 45 or 46, wherein the retinoid X receptor (RXR) or peroxisome proliferator activated receptor-gamma (PPARgamma) agonist is retinal, or a retinal derivative, stereoisomer, analogue or metabolite.

49.-58. (canceled)

59. A method of reducing or decreasing an immune response, inflammation or an inflammatory response in a subject, comprising administering a retinoic acid receptor agonist, or a retinoid X receptor (RXR) or peroxisome proliferator activated receptor-gamma (PPARgamma) agonist, to the subject in an amount that reduces or decreases the immune response, inflammation or an inflammatory response in the subject.

60. The method of claim 59, wherein the retinoic acid receptor agonist is vitamin A, or a vitamin A derivative, analogue or metabolite.

61. The method of claim 60, wherein the vitamin A metabolite comprises retinoic acid, or a retinoic acid derivative, analogue or isomer.

62. The method of claim 60, wherein the retinoic acid derivative comprises an ester or an amide.

63. The method of claim 60, wherein the retinoic acid derivative comprises fenretinide or retinaldehyde.

64. The method of claim 60, wherein the retinoic acid analogue comprises 9-cis retinoic acid, 13-cis retinoic acid or all trans retinoic acid.

65. The method of claim 61, wherein the retinoic acid isomer comprises an arotinoid.

66. The method of claim 65, wherein the arotinoid comprises adapalene or tazarotene.

67. The method of claim 59, wherein the agonist is retinal, or a retinal derivative, stereoisomer, analogue or metabolite.

68. The method of claim 67, wherein the retinal derivative, stereoisomer, analogue or metabolite is an all-trans, 13-cis, 1-cis, 9-cis, 7-cis, 11,13-cis or 9,13-cis vitamin A aldehyde, or a hydrate, a hemiacetal or an acetal form.

69. The method of claim 67, wherein the retinal derivative, stereoisomer, analogue or metabolite is Retinal hydrate; Retinal methyl hemiacetal; Retinal ethyl hemiacetal; Retinal propyl hemiacetal; Retinal isopropyl hemiacetal; Retinal butyl hemiacetal; Retinal pentyl hemiacetal; Retinal octyl hemiacetal; Retinal benzyl hemiacetal; Retinal dimethyl acetal; Retinal diethyl acetal; Retinal dipropyl acetal; Retinal diisopropyl acetal; Retinal dibutyl acetal; Retinal dipentyl acetal; Retinal dioctyl acetal; Retinal dibenzyl acetal; Retinal propylene glycol hemiacetal or acetal; Retinal 1,2-O-isopropylidene glyceryl hemiacetal or acetal; Retinal 3-allyloxy-1,2-propanediol hemiacetal or acetal; Retinal phythyl hemiacetal; Retinal diphytyl acetal; Retinal dodecyl hemiacetal; or Retinal didodecyl acetal.

70. The method of claim 67, wherein the retinal derivative is 5,6-dioxo-5,6-seco-retinal, 5,6-dihydro-5,6-epoxy-retinal, or 4-oxoretinal.

71. The method of claim 67, wherein the subject is treated for an immune response, inflammation or an inflammatory response in the skeletal joints or gastro-intestinal tract.

72. The method of claim 67, wherein the subject the retinoic acid receptor agonist is administered into a skeletal joint or gastro-intestinal tract.

73. The method of claim 67, wherein the subject has or is at risk of having multiple sclerosis (MS), diabetes mellitus types I or II, rheumatoid arthritis (RA), juvenile rheumatoid arthritis, osteoarthritis, psoriatic arthritis, encephalomyelitis, myasthenia gravis, systemic lupus erythematosus (SLE), autoimmune thyroiditis, atopic dermatitis, eczematous dermatitis, psoriasis, Sjögren's Syndrome, intestinal inflammation, Crohn's disease, inflammatory bowel disease (IBD), ulcerative colitis, Celiac disease, aphthous ulcer, iritis, conjunctivitis, keratoconjunctivitis, asthma, allergic asthma, cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, polymyostitis, Wegener's granulomatosis, hepatitis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, immune-mediated infertility, autoimmune Addison's disease, pemphigus vulgaris, pemphigus foliaceus, dermatitis herpetiformis, autoimmune alopecia, Vitiligo, autoimmune hemolytic anemia, pernicious anemia, Guillain-Barre syndrome, Stiff-man syndrome, acute rheumatic fever, sympathetic ophthalmia, Goodpasture's syndrome, systemic necrotizing vasculitis, primary biliary cirrhosis or myelodysplastic syndrome.

74. The method of claim 67, further comprising administering the regulatory T cells of claim 23 or the culture of regulatory T cells of claims 25 or 26 to the subject.

75. The method of claim 59, wherein the amount of retinoic acid receptor agonist or retinoid X receptor (RXR) or peroxisome proliferator activated receptor-gamma (PPARgamma) agonist is approximately equivalent to physiological amounts of retinoic acid.

76.-79. (canceled)

80. A culture of dendritic cells, said dendritic cells treated with a retinoic acid receptor agonist or a retinoid X receptor (RXR) or peroxisome proliferator activated receptor-gamma (PPARgamma) agonist that stimulates or increases differentiation into regulatory dendritic cells.

81. A culture of dendritic cells, said dendritic cells treated with a retinoic acid receptor agonist or a retinoid X receptor (RXR) or peroxisome proliferator activated receptor-gamma (PPARgamma) agonist and an antigen.

82. The culture of claims 80 or 81, wherein the dendritic cells comprise spleen dendritic cells, mucosal dendritic cells, blood, peripheral blood cells, bone marrow monocyte-derived dendritic cells, or inducible dendritic cells.

83. The culture of claim 82, wherein the inducible dendritic cells comprise CD34+ progenitor derived dendritic cells.

84. The culture of claims 80 or 81, wherein the dendritic cells comprise CD8-dendritic cells, or CD4−/CD8− dendritic cells.

85.-90. (canceled)

91. A method of reducing or suppressing an immune response to an antigen in a subject, comprising administering the regulatory T cells, the culture of regulatory T cells, or the culture of dendritic cells of claims 80 or 81, into the subject in an amount that reduces or suppresses the immune response to the antigen.

92. The method of claim 91, wherein the cell or cell culture was obtained or derived from the same subject as the subject administered the cells or cell culture.

93. The method of claim 91, wherein the subject has or is at risk of having an undesirable, aberrant or pathologic adaptive immune response.

94. (canceled)

95. The method of claim 91, wherein the subject has or is at risk of having an acute or chronic inflammatory response.

96. (canceled)

97. The method of claim 91, wherein the subject has or is at risk of having an autoimmune disease.

98. The method of claim 91, wherein the subject has or is at risk of having transplant rejection or graft-versus-host disease.

99. The method of claim 91, wherein the subject has or is at risk of having an allogenic stem cell transplantation, a bone marrow transplantation or an organ or tissue transplantation.

100. The method of claim 91, wherein the antigen comprises a self-antigen.

101. The method of claim 91, wherein the antigen comprises a non-self antigen to which an immune response is undesirable.

102.-104. (canceled)

105. A method of reducing or suppressing IL-17 expression or production in a cell, comprising contacting cells with a retinoic acid receptor agonist, or a retinoid X receptor (RXR) or peroxisome proliferator activated receptor-gamma (PPARgamma) agonist, in an amount that reduces or suppresses IL-17 expression or production in the cells.

106. The method of claim 105, wherein the agonist is retinal, or a retinal derivative, stereoisomer, analogue or metabolite.

107. The method of claim 105, wherein the cell is a CD4+ T cell.

108. The method of claim 105, wherein the cells are contacted in vitro or in vivo.
